(19) United States
(12) Patent Application Publication
(10) Pub. No.: US 2004/0039001 A1
(43) Pub. Date: Feb. 26, 2004

(54) 2-guanidino-4-aryl-quinazoline

(76) Inventors: Rolf Gericke, Seeheim (DE); Norbert Beier, Reinheim (DE); Claudia Wilm, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/363,169

(22) PCT Filed: Aug. 13, 2001
(86) PCT No.: PCT/EP01/09325

(30) Foreign Application Priority Data
Sep. 5, 2000 (DE) 100-43-667.6

(51) Int. Cl. A61K 31/55; A61K 31/517; C 07D 4 3/02; C07D 239/72

(52) U.S. Cl. 514/266.4; 544/292; 514/217.06; 514/266.2; 544/284

(57) ABSTRACT

The invention relates to compounds of the formula I, in which Y is (II) or (III) and Ar, R¹, R², R⁵, R⁶, R⁷ and R⁸ are as defined above, and their salts and solvates, and to their use as NHE-3 inhibitors.

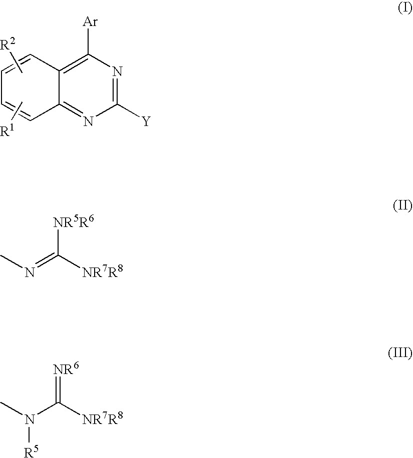

The invention relates to compounds of the formula I

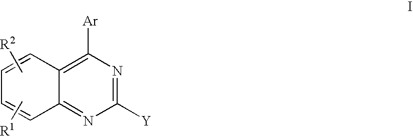

in which

Y is

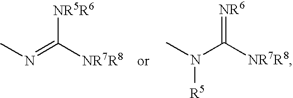

Ar is phenyl or naphthyl, each of which is unsubstituted or monosubstituted by R³ and/or R⁴,

R¹, R², R³ and R⁴ are each, independently of one another, H, A, OA, Hal, CF₃, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃,

A is alkyl having 1, 2, 3, 4, 5 or 6 carbon atoms,

Hal is F, Cl, Br or I

R⁵, R⁶, R⁷ and R⁸ are each, independently of one another, H, A, or phenyl which is unsubstituted or monosubstituted or polysubstituted by A, OA, Hal or CF₃,

where R⁵ and R⁷, R⁵ and R⁶, and R⁷ and R⁸ are able to form 5-7-membered rings,

and their salts and solvates, with the proviso that compounds in which R⁵, R⁶, R⁷ and R⁸ are simultaneously H and none of the radicals R¹, R², R³ and R⁴ is OH, NO₂, NH₂, NHA, NA₂, NH—CO-A, NH—CO-Ph, SA, SO-A, SO₂-A. SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃, are excluded.

The invention likewise relates to the use of the compounds of the formula I and their salts and solvates as NHE-3 inhibitors.

Other inhibitors of the sodium/proton exchanger subtype 3 have already been described, for example in EP 0 825 178.

The compounds excepted by the proviso have already been described in U.S. Pat. No. 3,131,187, as has their use for other purposes.

Quinazolinylguanidine derivatives have been described by V. I. Shvedov et al. in Pharm. Chem. J. (Engl. transl.) 1980, 14, 532-538 or in Khim. Farm. Zh. 1980, 14, 38-43, and by S. C. Bell et al. in J. Med. Pharm. Chem. 1962, 5, 63-69.

The invention had the object of finding novel compounds having valuable properties, in particular those which can be used for the preparation of medicaments.

Surprisingly, it has been found that the compounds of the formula I and their salts are well tolerated and inhibit sodium/proton exchanger subtype 3.

The compounds of the formula I can be employed as medicament active ingredients in human and veterinary medicine.

It is known that the Na⁺/H⁺ exchanger represents a family having at least six different isoforms (NHE-1 to NHE-6), all of which have already been cloned. While subtype NHE-1 is distributed ubiquitously in all tissues throughout the body, the other NHE subtypes are expressed selectively in specific organs, such as in the kidney or in the lumen wall and contra-luminal wall of the small intestine. This distribution reflects the specific functions that the various isoforms serve, namely on the one hand regulation of the intracellular pH and cell volume by subtype NHE-1 and on the other hand Na⁺ absorption and resorption in the intestine and kidney by isoforms NHE-2 and NHE-3. Isoform NHE-4 has been found principally in the stomach. Expression of NHE-5 is restricted to the brain and neuronal tissue. NHE-6 is the isoform that forms the sodium/proton exchanger in the mitochondria.

The isoform NHE-3 is expressed in particular in the apical membrane of the proximal renal tubuli: an NHE-3 inhibitor therefore exerts, inter alia, a protective action on the kidneys.

The therapeutic use of a selective inhibitor for NHE-3 isoforms is manifold. NHE-3 inhibitors inhibit or reduce tissue damage and cell necrosis after pathophysiological hypoxic and ischaemic events which result in activation of the NHE activity, as is the case during renal ischaemia or during the removal, transport and reperfusion of a kidney during a kidney transplant. The compounds of the formula I have a cytoprotective action in that they prevent the excessive absorption of sodium and water into the cells of organs undersupplied with oxygen.

The compounds of the formula I have a hypotensive action and are suitable as medicament active ingredients for the treatment of hypertonia. They are furthermore suitable as diuretics.

The compounds of the formula I, alone or in combination with NHE inhibitors of other subtype specificity, have an antiischaemic action and can be used in the case of thromboses, atherosclerosis, vascular spasms, for the protection of organs, for example kidney and liver, before and during operations, and in the case of chronic or acute renal failure.

They can furthermore be used for the treatment of strokes, cerebral oedema, ischaemia of the nervous system, various forms of shock, for example allergic, cardiological, hypovolemic or bacterial shock, and for improving breathing drive in, for example, the following states: central sleep apnoea, cot death, postoperative hypoxia and other breathing disorders.

Through combination with a carboanhydrase inhibitor, breathing activity can be further improved.

The compounds of the formula I have an inhibiting effect on the proliferation of cells, for example fibroblast cell proliferation and the proliferation of the smooth muscle cells, and can therefore be used for the treatment of illnesses in which cell proliferation is a primary or secondary cause. The compounds of the formula I can be used against delayed complications of diabetes, cancer illnesses, fibrotic illnesses, endothelial dysfunction, organ hypertrophia and hyperplasia, in particular in prostate hyperplasia or prostate hypertrophia.

They are furthermore suitable as diagnostic agents for the determination and differentiation of certain forms of hypertonia, atherosclerosis, diabetes and proliferative illnesses.

Since the compounds of the formula I also have an advantageous effect on the level of serum lipoproteins, they can be employed, alone or in combination with other medicaments, for the treatment of an increased blood fat level.

The invention relates to the use of compounds of the formula I according to claim 1 and their physiologically acceptable salts and/or solvates for the preparation of a medicament for the treatment of thrombosis, ischaemic states of the heart, of the peripheral and central nervous system and of strokes, ischaemic states of peripheral organs and extremities and for the treatment of shock states.

The invention furthermore relates to the use of compounds of the formula I according to claim 1 and their physiologically acceptable salts and/or solvates for the preparation of a medicament for use in surgical operations and organ transplants and for the preservation and storage of transplants for surgical measures.

The invention also relates to the use of compounds of the formula I according to claim 1 and their physiologically acceptable salts and/or solvates for the preparation of a medicament for the treatment of illnesses in which cell proliferation is a primary or secondary cause, for the treatment or prophylaxis of disorders of fat metabolism or disturbed breathing drive.

The invention furthermore relates to the use of compounds of the formula I according to claim 1 and their physiologically acceptable salts and/or solvates for the preparation of a medicament for the treatment of renal ischaemia, ischaemic intestinal illnesses or for the prophylaxis of acute or chronic renal illnesses.

Methods for the identification of substances which inhibit sodium/proton exchanger subtype 3 are described, for example, in U.S. Pat. No. 5,871,919.

The compounds of the formula I are, in addition, suitable for the treatment of bacterial and parasitic illnesses.

For all radicals in the compounds of the formula I which occur more than once, such as, for example, A, their meanings are independent of one another.

The term hydrates is taken to mean, for example, the hemi-, mono- or dihydrates, and the term solvates is taken to mean, for example, alcohol addition compounds, such as, for example, with methanol or ethanol.

In the formulae above, A is alkyl is linear or branched, and has 1, 2, 3, 4, 5 or 6 carbon atoms. A is preferably methyl, furthermore ethyl, propyl, iso-propyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, or 1,1,2- or 1,2,2-trimethylpropyl.

OA is preferably methoxy, ethoxy, propoxy, isopropoxy or butoxy.

Hal is preferably F, Cl or Br, but also I, in particular F, Cl or Br.

Above and below, Ph is an unsubstituted phenyl radical unless stated otherwise.

Ar is preferably unsubstituted phenyl or naphthyl, furthermore preferably phenyl or naphthyl which is monosubstituted, for example, by A, fluorine, chlorine, bromine, iodine, methoxy, ethoxy, propoxy, butoxy or CF₃. Ar is particularly preferably phenyl which is unsubstituted or mono-substituted by A, fluorine, chlorine, bromine, iodine, methoxy, ethoxy, propoxy, butoxy or CF₃.

R⁵, R⁶, R⁷ and R⁸ are preferably simultaneously H or, independently of one another, H or A, which is as defined above.

If R⁵ and R⁷ together form a ring, Y preferably adopts one of the following structures:

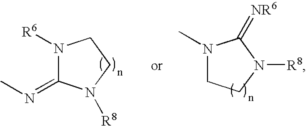

in which R⁶ and R⁸ are as defined above, and n is 1, 2 or 3, preferably 1 or 2.

If R⁷ and R⁸ together form a ring, Y preferably adopts one of the following structures:

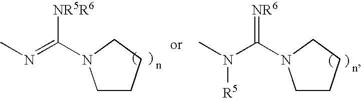

in which R⁵ and R⁶ are as defined above, and n is 1, 2 or 3, preferably 1 or 2.

If R⁵ and R⁶ together form a ring, Y preferably adopts one of the following structures:

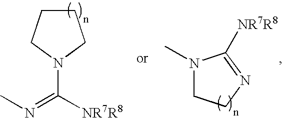

in which R⁷ and R⁸ are as defined above, and n is 1, 2 or 3, preferably 1 or 2.

Accordingly, the invention relates in particular to the use of the compounds of the formula I in which at least one of the said radicals has one of the preferred meanings indicated above, and to the use thereof. Some preferred groups of compounds may be expressed by the following sub-formulae Ia to Ie, which conform to the formula i and in which the radicals not designated in greater detail have the meaning indicated in the formula but in which

| | | |
|---|---|---|
| in Ia | R¹ | is H, OH, OA, SA or Hal, in particular H, OH, OCH₃ or |
| | | CH₃; |
| in Ib | R¹ | is H, OH, OA, SA or Hal, in particular H, OH, OCH₃ or |
| | | CH₃, |
| | R2 | is H, Hal, OH, A, NH₂, NO₂ or CN, in particular H, Cl, |
| | | OH, CH₃ or NH₂; |
| in Ic | R¹ | is H, OH, OA, SA or Hal, in particular H, OH, OCH₃ or |
| | | CH₃, |
| | R² | is H, Hal, OH, A, NH₂, NO₂ or CN, in particular H, Cl, |
| | | OH, CH₃ or NH₂, |
| | Ar | is phenyl; |
| in Id | R¹ | is H, OH, OA, SA or Hal, in particular H, OH, OCH₃ or |
| | | CH₃, |
| | R² | is H, Hal, OH, A, NH₂, NO₂ or CN, in particular H, Cl, |
| | | OH, CH₃ or NH₂, |
| | Ar | is phenyl, |
| | R³ | is H, A, NH₂ or SA, in particular H or CH₃; |
| in Ie | R¹ | is H, OH, OA, SA or Hal, in particular H, OH, OCH₃ or |
| | | CH₃, |
| | R² | is H, Hal, OH, A, NH₂, NO₂ or CN, in particular H, Cl, |
| | | OH, CH₃ or NH₂, |
| | Ar | is phenyl, |
| | R³ | is H, A, NH₂ or SA, in particular H or CH₃, |
| | R⁴ | is H, Hal, NH₂ or NO₂, in particular H or NH₂. |

Preference is further given to compounds of the formula I and their salts and solvates in which R is simultaneously H. Ar is phenyl and at least one of the radicals R¹, R², R³ and R⁴ have one of the following meanings: OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO-NH₂, CO—NHA, CO—NA₂, SO₂NH₂, SO₂NHA, SO₂NA₂, or phenyl which is unsubstituted or monosubstituted or polysubstituted by A, OA, Hal or CF₃. Of these compounds, particular preference is given to those whose radical R¹ is Cl, in particular in position 6, and those compounds whose radical R³ is methyl, in particular in position 4′.

Preference is also given to compounds of the formula I and their salts and solvates in which the radicals R⁵, R⁶, R⁷ and R⁸ are simultaneously H. Of these compounds, particular preference is given to those whose radical R¹ is Cl, in particular in position 6, and compounds whose radical R³ is methyl, in particular in position 4′, and compounds whose radical R⁴ is NH₂, in particular in position 2′.

Compounds of the formula I whose radical R³ is methyl, in particular in position 4′, have a particularly pronounced selectivity of the binding to the NHE-3 receptor.

Compounds of the formula I whose radical R⁴ is NH₂, in particular in position 2′, exhibit particularly good solubility in aqueous solutions.

Compounds of the formula I in which R¹ is H, R² is Cl in position 6 and R³ is methyl in position 4′ are preferred. Very particular preference is given to compounds of the formula I whose radical R⁴ is additionally NH₂ in position 2′.

Particular preference is given to the compounds of the formulae If to Ik:

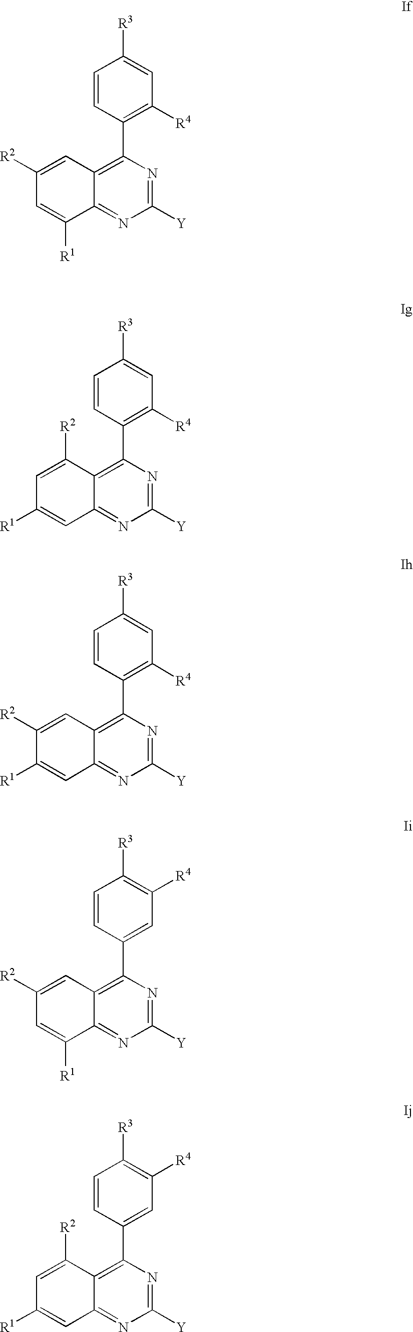

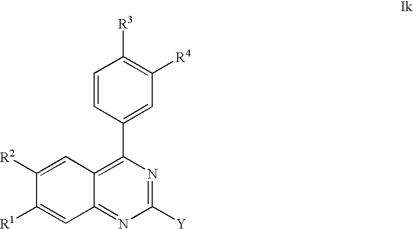

in which R¹, R², R³, R⁴ and Y are as defined above, and R¹ is preferably H. OH, OA, SA or F, in particular H. OH, OCH₃ or CH₃, R¹ in the formulae If to Ik is very particularly preferably H.

R² is preferably H, Cl, A, NH₂, NO₂, SCH₃, SOCH₃, SO₂CH₃, OCH₃, OH, CN, CF₃, OCF₃ or F, in particular H, Cl, F, Br, OH, CH₃, NO₂ or NH₂. R² in the formulae If to Ik is very particularly preferably Cl.

R³ is preferably H, Cl, A, NH₂, NO₂, SCH₃, CN, C₂H₅, OCF₃ or C₆H₅, in particular H. A or CH₃, R³ in the formula If to Ik is very particularly preferably CH₃.

R⁴ is preferably H, F, NH₂ or NO₂, in particular H or NH₂. R⁴ in the formulae If to Ik is very particularly preferably NH₂.

Y in the formulae If to Ik is as defined above. Y therein preferably adopts one of the following meanings.

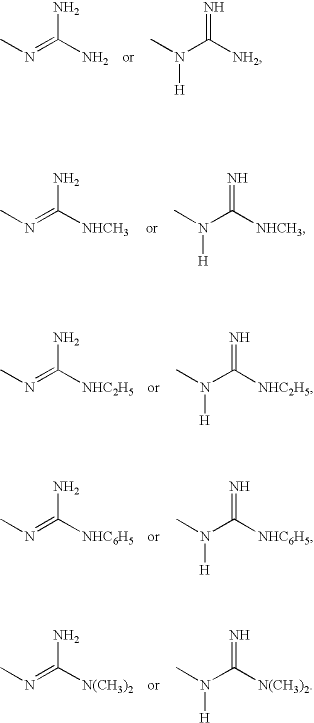

Y particularly preferably has one of the following meanings:

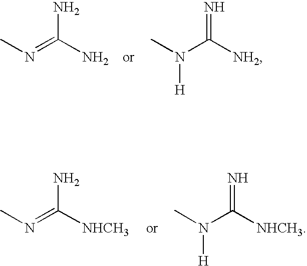

Particularly preference is furthermore given to the following compounds I1 to I10 and their salts and solvates:

| | |
|---|---|
| N-(6-chloro-4-phenylquinazolin-2-yl)-N′-methylguanidine | I1 |
| N-(6-chloro-4-p-tolylquinazolin-2-yl)-N′-methylguanidine | I2 |
| N-[6-chioro-4-(2-nitrophenyl)quinazolin-2-yl]-N′-methyl- | I3 |
| guanidine | |
| N-[4-(2-aminophenyl)-6-chloroquinazolin-2-yl]-N′-methyl- | I4 |
| guanidine | |
| N-[6-chloro-4-(4-methyl-2-nitrophenyl)quinazolin-2-yl]-N′- | I5 |
| methylguanidine | |
| N-(4-(2-amino-4-methylphenyl)-6-chloroquinazolin-2-yl]-N′- | I6 |
| methylguanidine | |
| N-[6-chloro-4-(2-nitrophenyl)quinazolin-2-yl]guanidine | I7 |
| N-[4-(2-aminophenyl)-6-chloroquinazolin-2-yl]guanidine | I8 |
| N-[6-chloro-4-(4-methyl-2-nitrophenyl)quinazolin-2-yl]- | I9 |
| guanidine | |
| N-[4-(2-amino-4-methylphenyl)-6-chloroquinazolin-2-yl]- | I10 |
| guanidine | |

The hydrochlorides and p-toluenesulfonates of the compounds of the formulae I1 to I10 are very particularly preferred.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

The starting materials can, if desired, also be formed in situ, so that they are not isolated from the reaction mixture, but instead are immediately converted further into the compounds of the formula I.

The 2guanidineo-4-arylquinazolines of the formula I are preferably prepared by reacting o-aminophenyl ketones o-aminonaphthyl ketones of the formula II

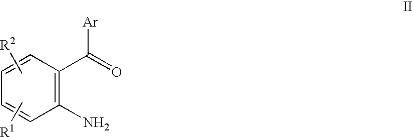

in which R¹, R² and Ar are as defined in claim 1, with 1-cyanoguanidine or a correspondingly N-alkylated or N-arylated 1-cyanoguanidine of the formula NC—Y, in which Y is as defined above.

The reaction can be carried out in an inert solvent.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

DMF, water or an alcohol is preferably used.

The reaction is very particularly preferably carried out without a solvent, i.e. in the melt, at temperatures between 100 and 200° C.

Of advantage is the presence of an acidic catalyst, such as AlCl₃, TiCl₄, p-toluenesulfonic acid, BF₃, acetic acid, sulfuric acid, oxalic acid, POCl₃ or phosphorus pentoxide.

A preferred variant comprises employing one of the reactants already as a salt, for example as the hydrochloride.

A further valuable method for the preparation of the compounds of the formula I comprises reacting, instead of a compound of the formula NC—Y, a compound of the formula III

HN═CX—Y III

in which

x is —S-alkyl, —S-aryl, O-alkyl or O-aryl,

and alkyl is preferably as defined above for A, and aryl is preferably as defined above for Ar,

with a compound of the formula II.

Finally, the compounds of the formula I can be prepared by reaction of 2-chloro-4-arylquinazolines of the formula IV

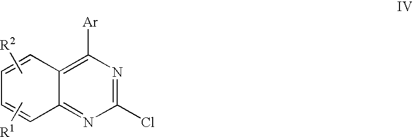

in which Ar, R¹ and R² are as defined above,

with a compound of the formula HY, in which Y is as defined above. HY is particularly preferably guanidine.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethane-disulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

The invention furthermore relates to the use of the compounds of the formula I as NHE-3 inhibitors and/or their physiologically acceptable salts for the preparation of pharmaceutical preparations, in particular by non-chemical methods. In this case, they can be converted into a suitable dosage form together with at least one solid, liquid and/or semiliquid excipient or assistant, and, if desired, in combination with one or more further active ingredients.

The invention furthermore relates to pharmaceutical preparations comprising at least one NHE-3 inhibitor of the formula I and/or one of its physiologically acceptable salts and solvates.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders, or transdermally in patches.

The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins.

Suitable pharmaceutical preparations for administration in the form of aerosols or sprays are, for example, solutions, suspensions or emulsions of the active ingredient of the formula I in a pharmaceutically acceptable solvent.

The compounds of the formula I and their physiologically acceptable salts and solvates can be used for the treatment and/or prophylaxis of the illnesses or illness states described above.

In general, the substances according to the invention are preferably administered in doses between about 0.1 and 500 mg, in particular between 1 and 10 mg, per dosage unit. The daily dose is preferably between about 0.001 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

### EXAMPLES

### Example 1

A mixture of 1.00 g of 2-amino-5-chloro-2′-nitrobenzophenone, 0.60 g of 1-cyanoguanidine and 2.00 g of p-toluenesulfonic acid monohydrate was melted at 150° C. for 2 hours. Methanol was added to the cooled melt, and the mixture was stirred at 65° C. for 30 minutes. The residue obtained after filtration was discarded, and water was added to the filtrate. The solution was subsequently rendered alkaline and extracted with ethyl acetate. The extract was evaporated and crystallised from acetonitrile, giving the free base N-[6-chloro-4-(2-nitrophenyl)quinazolin-2-yl]guanidine.

In order to form the acid-addition salt, the base was dissolved in methanol, the mixture was acidified using HCl-containing isopropanol, and the solvent was subsequently removed, Crystals of N-[6-chloro-4-(2-nitrophenyl)-quinazolin-2-yl]guanidinium chloride were obtained from acetonitrile.

### Example 2

1.20 g of N-(5-methoxy-4-phenylquinazolin-2-yl)guanidinium chloride were stirred at 170° C. for 6 hours with 8.00 g of pyridinium chloride. The cooled melt was subsequently treated with 20 ml of an Na₂S₂O₄ solution. The resultant precipitate was isolated and dissolved in methanol, and the solution was acidified using HCl-containing isopropanol. After the solvent had been removed, the residue was crystallised from acetonitrile, giving N-(5-hydroxy-4-phenylquinazolin-2-yl)guanidinium chloride (m.p. 310° C.).

### Example 3

A mixture of 3.01 g of 2-amino-5-chlorobenzophenone, 2.55 g of N-cyano-N′-methylguanidine and 7.42 g of p-toluenesulfonic acid monohydrate was stirred in the melt at from 150 to 160° C. for 2 hours. Methanol was added to the cooled melt, and the mixture was stirred at 65° C. for 30 minutes. The residue obtained after filtration was discarded, water and ethyl acetate were added to the filtrate, and the mixture was again stirred at 65° C. for 30 minutes. The product was subsequently allowed to crystallise out with stirring in an ice bath, giving N-(6-chloro-4-phenylquinazolin-2-yl)-N′-methylguanidinium p-toluenesulfonate (m.p. 268-269° C.).

### Example 4

300 mg of N-[6-chloro-4-(2-nitrophenyl)quinazolin-2-yl]guanidinium p-toluenesulfonate were dissolved in 50 ml of methanol and hydrogenated at RT over the course of 21 hours at atmospheric-pressure in the pressure of 300 mg of Raney nickel. Filtration and removal of the solvent gave N-[6-chloro-4-(2-aminophenyl)quinazolin-2-yl]guanidinium p-toluenesulfonate from the filtrate. (m.p. 250° C.).

### Example 5

A mixture of 0.350 g of N-(6-methylsulfanyl-4-phenylquinazolin-2-yl)-guanidinium chloride and 0.140 g of sodium perborate trihydrate in 5 ml of acetic acid was stirred at 80° C. for 30 minutes. The solution was subsequently evaporated and water was added. The aqueous solution was adjusted to pH 12 and extracted with ethyl acetate. Evaporation of the extract gave N-(6-methanesulfinyl-4-phenylquinazolin-2-yl)guanidine in crystalline form (m.p. 175-180° C.).

### Example 6

A mixture of 1.200 g of N-(6-methylsulfanyl-4-phenylquinazolin-2-yl)-guanidinium chloride and 0.154 g of sodium perborate trihydrate in 5 ml of acetic acid was stirred at 80° C. for 1 hour. The reaction mixture was subsequently evaporated, and water was added. The resultant solution was adjusted to pH 12 and extracted with ethyl acetate. Evaporation of the extract gave N-(6-methanesulfonyl-4-phenylquinazolin-2-yl)guanidine in crystalline form (m.p. 180-185° C.).

In order to form the acid-addition salt, 0.80 g of N-(6-methanesulfonyl-4-phenylquinazolin-2-yl)guanidine were treated with an aqueous 1 N HCl solution, and the resultant crystals were recrystallised from ethanol.

### Example 7

2.70 g of the hydrochloride of 2-amino-5-chlorobenzophenone and 1.70 g of N-cyano-N′,N′″-dimethylguanidine were mixed and heated at 150° C. for 3 hours. The reaction product was taken up in methanol and filtered. The filtrate was evaporated. The residue was recrystallised from a mixture of isopropanol and diethyl ether, giving N-(6-chloro-4-phenylquinazolin-2-yl)-N′,N″-dimethylguanidinium chloride (m.p. 264-267° C.).

### Example 8

A mixture of 500 mg of 2-amino-5-chloro-2′-nitrobenzophenone, 406 mg of N-cyano-N′-ethylguanidine and 1.03 g of p-toluenesulfonic acid mono-hydrate was stirred in the melt at from 150 to 160° C. for 2 hours and worked up as in Example 3, giving N-[6-chloro-4-(2-nitrophenyl)quinazolin-2-yl]-N′-ethylguanidinium p-toluenesulfonate (m.p. 298-300° C.).

### Example 9

A mixture of 500 mg of 2-amino-5-chloro-2′-nitrobenzophenone, 580 mg of N-cyano-N-phenylguanidine and 1.03 g of p-toluenesulfonic acid monohydrate was stirred in the melt at from 150 to 160° C. for 2 hours and worked up as in Example 3 giving N-[6-chloro-4-(2-nitrophenyl)quinazolin-2-yl]-N′-phenylguanidinium p-toluenesulfonate (m.p. 261-263° C.).

The following acid-addition salts which are preferred as NHE-3 inhibitors were obtained analogously to the above-mentioned processes using the corresponding precursors:

pTsOH denotes p-toluenesulfonic acid.

### Examples 10-101

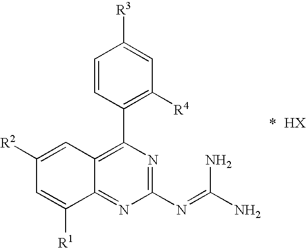

| | R¹ | R² | R³ | R⁴ | HX | |
|---|---|---|---|---|---|---|
| (10) | H | Cl | H | SO₂CH₃ | pTsOH | |
| (11) | H | Cl | CH₃ | SO₂CH₃ | HCl | |
| (12) | H | Cl | C₂H₅ | SO₂CH₃ | HCl | |
| (13) | H | Cl | OCH₃ | SO₂CH₃ | HCl | |
| (14) | H | Cl | NO₂ | H | pTsOH | |
| (15) | H | Cl | NH₂ | H | pTsOH | (m.p.260-266° C.) |
| (16) | H | Cl | N(CH₃)₂ | H | pTsOH | |
| (17) | H | Cl | H | NH₂ | HCl | |
| (18) | H | Cl | CH₃ | NH₂ | pTsOH | (m.p.211-214° C.) |
| (19) | H | Cl | C₂H₅ | NH₂ | HCl | |
| (20) | H | Cl | OCH₃ | NH₂ | HCl | |
| (21) | H | Cl | NO₂ | NH₂ | HCl | |
| (22) | H | Cl | NH₂ | NH₂ | HCl | |
| (23) | H | Cl | N(CH₃)₂ | NH₂ | HCl | |
| (24) | H | Cl | H | NHCH₃ | HCl | |
| (25) | H | Cl | CH₃ | NHCH₃ | HCl | |
| (26) | H | Cl | C₂H₅ | NHCH₃ | HCl | |
| (27) | H | Cl | OCH₃ | NHOH₃ | HCl | |
| (28) | H | Cl | NO₂ | NHCH₃ | HCl | |
| (29) | H | Cl | NH₂ | NHCH₃ | HCl | |
| (30) | H | Cl | N(CH₃)₂ | NHCH₃ | HCl | |
| (31) | H | Cl | H | N(CH₃)₂ | HCl | |
| (32) | H | Cl | CH₃ | N(CH₃)₂ | HCl | |
| (33) | H | Cl | C₂H₅ | N(CH₃)₂ | HCl | |
| (34) | H | Cl | OCH₃ | N(CH₃)₂ | HCl | |
| (35) | H | 01 | NO₂ | N(CH₃)₂ | HCl | |
| (36) | H | Cl | NH₂ | N(CH₃)₂ | HCl | |
| (37) | H | Cl | N(CH₃)₂ | N(CH₃)₂ | HCl | |
| (38) | H | Cl | H | OH | HCl | |
| (39) | H | Cl | OH₃ | OH | HCl | |
| (40) | H | Cl | C₂H₅ | OH | HCl | |
| (41) | H | Cl | OCH₃ | OH | HCl | |
| (42) | H | Cl | NO₂ | OH | HCl | |
| (43) | H | Cl | NH₂ | OH | HCl | |
| (44) | H | Cl | N(CH₃)₂ | OH | HCl | |
| (45) | H | Cl | SO₂CH₃ | CH₃ | HCl | |
| (46) | H | Cl | H | CN | HCl | (m.p. >350°, |
| | | | | | | decomposition |
| (47) | H | Cl | C₂H₅ | SO₂NH₂ | HCl | |
| (48) | H | Cl | OCF₃ | CH₃ | HCl | |
| (49) | H | Cl | NO₂ | CH₃ | HCl | |
| (50) | H | Cl | NH₂ | CH₃ | HCl | |
| (51) | H | Cl | N(CH₃)₂ | CH₃ | HCl | |
| (52) | H | Cl | H | NO₂ | pTsOH | (m.p. 313-315° C.) |
| (53) | H | Cl | NO₂ | H | HCl | (m.p. 346° C.) |
| (54) | H | H | NH₂ | H | HCl | |
| (55) | H | H | NH₂ | CH₃ | HCl | |
| (56) | H | Cl | OH₃ | CO—NH₂ | HCl | |
| (57) | H | H | OH₃ | SO₂CH₃ | pTsOH | |
| (58) | H | Cl | OH | F | pTsOH | |
| (59) | H | Cl | F | SCH₃ | HCl | |
| (60) | H | Br | H | CONH₂ | pTsOH | |
| (61) | H | Br | CO—NH₂ | F | pTsOH | |
| (62) | H | NO₂ | H | H | pTsOH | (m.p. 317-320° C.) |
| (63) | H | OCH₃ | H | OCF₃ | pTsOH | |
| (64) | H | OH | H | H | HCl | (m.p. 333° C.) |
| (65) | H | NH₂ | H | H | HCl | (m.p. 290-296° C.) |
| (66) | H | SCH₃ | H | H | HCl | (m.p. 234-238° C.) |
| (67) | H | CH₃ | CN | CO—NH₂ | pTsOH | |
| (68) | H | C₆H₅ | H | H | pTsOH | (m.p. 188° C.) |
| (69) | H | CF₃ | SOCH₃ | H | HCl | |
| (70) | H | OCF₃ | H | H | HCl | (m.p. 255-259° C.) |
| (71) | H | CN | H | H | HCl | (m.p. 330° C.) |
| (72) | H | F | H | SOC₂H₅ | pTsOH | |
| (73) | H | SOCH₃ | H | H | pTsOH | |
| (74) | H | SO₂CH₃ | H | H | pTsOH | |
| (75) | H | Cl | CN | H | HCl | (m.p. 344° C.) |
| (76) | NH₂ | Cl | Cl | Cl | HCl | |
| (77) | H | Cl | H | OCF₃ | pTsOH | (m.p. 274-277° C.) |
| (78) | H | Cl | OCF₃ | H | HCl | (m.p. 310-315° C.) |
| (79) | Cl | Cl | CH₃ | OH | HCl | |
| (80) | Cl | H | NH₂ | H | HCl | |
| (81) | Cl | H | NH₂ | OH₃ | HCl | |
| (82) | OH₃ | Cl | CH₃ | CO₂H | HCl | |
| (83) | C₆H₅ | Cl | CH₃ | F | HCl | |
| (84) | OH | CO—NH₂ | H | H | pTsOH | |
| (85) | Cl | H | H | SCH₃ | pTsOH | |
| (86) | H | Cl | Cl | SCH₃ | pTsOH | |
| (87) | SCH₃ | H | H | H | HCl | (m.p. 303-306° C.) |
| (88) | H | F | CH₃ | CN | HCl | |
| (89) | H | Cl | SCH₃ | H | HCl | (m.p. 324-327° C.) |
| (90) | CH₃ | H | CN | H | HCl | |
| (91) | H | Cl | C₆H₅ | H | HCl | (m.p. 200° C.) |
| (92) | H | Cl | CH₃ | NO₂ | pTsOH | (m.p. 210-214° C.) |
| (93) | H | H | Br | SO₂CH₃ | pTsOH | |
| (94) | H | H | OCH₃ | OCF₃ | pTsOH | |
| (95) | H | Cl | H | CN | HCl | (m.p. >350° C., |
| | | | | | | decomposition) |
| (96) | H | Cl | C₂H₅ | NH₂ | pTsOH | (m.p. >257° C., |
| | | | | | | decomposition |
| (97) | H | Cl | CF₃ | NO₂ | pTsOH | (m.p. 304-308° C.) |
| (98) | H | Cl | C₂H₅ | NO₂ | pTsOH | (m.p. 286-287° C.) |
| (99) | H | Cl | SOCH₃ | H | HCl | (m.p. 322-324° C.) |
| (100) | H | Cl | CF₃ | NH₂ | pTsOH | (m.p. >232° C.) |
| (101) | H | Cl | N(C₂H₅)₂ | H | HCl | (m.p. 200° C.) |

### Examples 102-154

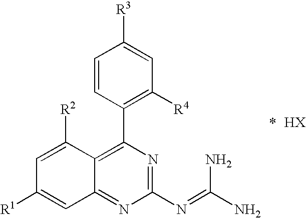

| | R¹ | R² | R³ | R⁴ | HX | |
|---|---|---|---|---|---|---|
| (102) | H | Cl | H | SO₂CH₃ | pTsOH | |
| (103) | H | Cl | CH₃ | SO₂CH₃ | HCl | |
| (104) | H | Cl | C₂H₅ | SO₂CH₃ | HCl | |
| (105) | H | Cl | OCH₃ | SO₂CH₃ | HCl | |
| (106) | H | Cl | NO₂ | H | HCl | |
| (107) | H | Cl | NH₂ | H | HCl | |
| (108) | H | Cl | N(CH₃)₂ | H | HCl | |
| (109) | H | Cl | H | NH₂ | HCl | |
| (110) | H | Cl | CH₃ | NH₂ | HCl | |
| (111) | H | Cl | C₂H₅ | NH₂ | HCl | |
| (112) | H | Cl | OCH₃ | NH₂ | HCl | |
| (113) | H | Cl | NO₂ | NH₂ | HCl | |
| (114) | H | Cl | NH₂ | NH₂ | HCl | |
| (115) | H | Cl | N(CH₃)₂ | NH₂ | HCl | |
| (116) | H | Cl | H | NHCH₃ | HCl | |
| (117) | H | Cl | CH₃ | NHCH₃ | HCl | |
| (118) | H | Cl | C₂H₅ | NHCH₃ | HCl | |
| (119) | H | Cl | OCH₃ | NHCH₃ | HCl | |
| (120) | H | Cl | NO₂ | NHCH₃ | HCl | |
| (121) | H | Cl | NH₂ | NHCH₃ | HCl | |
| (122) | H | Cl | N(CH₃)₂ | NHCH₃ | HCl | |
| (123) | H | Cl | H | N(CH₃)₂ | HCl | |
| (124) | H | Cl | CH₃ | N(CH₃)₂ | HCl | |
| (125) | H | Cl | C₂H₅ | N(CH₃)₂ | HCl | |
| (126) | H | Cl | OCH₃ | N(CH₃)₂ | HCl | |
| (127) | H | Cl | NO₂ | N(CH₃)₂ | HCl | |
| (128) | H | Cl | NH₂ | N(CH₃)₂ | HCl | |
| (129) | H | Cl | N(CH₃)₂ | N(CH₃)₂ | HCl | |
| (130) | H | Cl | H | OH | HCl | |
| (131) | H | Cl | CH₃ | OH | HCl | |
| (132) | H | Cl | C₂H₅ | OH | HCl | |
| (133) | H | Cl | OCH₃ | OH | HCl | |
| (134) | H | Cl | NO₂ | OH | HCl | |
| (135) | H | Cl | NH₂ | OH | HCl | |
| (136) | H | Cl | N(CH₃)₂ | OH | HCl | |
| (137) | H | Cl | SCH₃ | CH₃ | HCl | |
| (138) | H | Cl | CH₃ | CH₃ | HCl | |
| (139) | H | Cl | C₂H₅ | CH₃ | HCl | |
| (140) | H | Cl | OCH₃ | CH₃ | HCl | |
| (141) | H | Cl | NO₂ | CH₃ | HCl | |
| (142) | H | Cl | NH₂ | CH₃ | HCl | |
| (143) | H | Cl | N(CH₃)₂ | CH₃ | HCl | |
| (144) | H | OCF₃ | NH₂ | H | HCl | |
| (145) | H | OCF3 | NH₂ | OH₃ | HCl | |
| (146) | H | OCH₃ | SO₂CH₃ | SO₂CH₃ | pTsOH | |
| (147) | H | OH | H | H | pTsOH | |
| (148) | Cl | OCH₃ | NH₂ | H | HCl | |
| (149) | Cl | Cl | NH₂ | OH₃ | HCl | |
| (150) | OCH₃ | SCH₃ | H | H | pTsOH | |
| (151) | OH | H | H | H | HCl | (m.p. |
| | | | | | | 326° C.) |
| (152) | Cl | F | H | CONH₂ | pTsOH | |
| (153) | H | CH₃ | n-SC₅H₁₁ | H | pTsOH | |
| (154) | H | Cl | SO₂NH₂ | F | pTsOH | |

### Examples 155-205

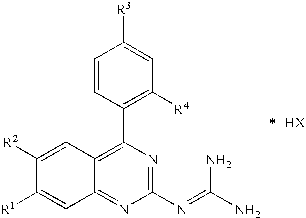

| | R¹ | R² | R³ | R⁴ | HX | |
|---|---|---|---|---|---|---|
| (155) | OH | Cl | H | SO₂CH₃ | HCl | |
| (156) | OH | Cl | CH₃ | SO₂CH₃ | HCl | |
| (157) | OH | Cl | C₂H₅ | SO₂CH₃ | HCl | |
| (158) | OH | Cl | OCH₃ | SO₂CH₃ | HCl | |
| (159) | OH | Cl | NO₂ | H | HCl | |
| (160) | OH | Cl | NH₂ | H | HCl | |
| (161) | OH | Cl | N(CH₃)₂ | H | HCl | |
| (162) | OH | Cl | H | NH₂ | HCl | |
| (163) | OH | Cl | CH₃ | NH₂ | HCl | |
| (164) | OH | Cl | C₂H₅ | NH₂ | HCl | |
| (165) | OH | Cl | OCH₃ | NH₂ | HCl | |
| (166) | OH | Cl | NO₂ | NH₂ | HCl | |
| (167) | OH | Cl | NH₂ | NH₂ | HCl | |
| (168) | OH | Cl | N(CH₃)₂ | NH₂ | HCl | |
| (169) | OH | Cl | H | NHCH₃ | HCl | |
| (170) | OH | Cl | CH₃ | NHCH₃ | HCl | |
| (171) | OH | Cl | C₂H₅ | NHCH₃ | HCl | |
| (172) | OH | Cl | OCH₃ | NHCH₃ | HCl | |
| (173) | OH | Cl | NO₂ | NHCH₃ | HCl | |
| (174) | OH | Cl | NH₂ | NHCH₃ | HCl | |
| (175) | OH | Cl | N(CH₃)₂ | NHCH₃ | HCl | |
| (176) | OH | Cl | H | N(CH₃)₂ | HCl | |
| (177) | OH | Cl | CH₃ | N(CH₃)₂ | HCl | |
| (178) | OH | Cl | C₂H₅ | N(CH₃)₂ | HCl | |
| (179) | OH | Cl | OCH₃ | N(CH₃)₂ | HCl | |
| (180) | OH | Cl | NO₂ | N(CH₃)₂ | HCl | |
| (181) | OH | Cl | NH₂ | N(CH₃)₂ | HCl | |
| (182) | OH | Cl | N(CH₃)₂ | N(CH₃)₂ | HCl | |
| (183) | OH | Cl | H | OH | OH | |
| (184) | OH | Cl | OH₃ | OH | OH | |
| (185) | OH | Cl | C₂H₅ | OH | OH | |
| (186) | OH | Cl | C₂H₅ | OH | OH | |
| (186) | OH | Cl | OCH₃ | OH | OH | |
| (187) | OH | Cl | NO₂ | OH | OH | |
| (188) | OH | Cl | NH₂ | OH | OH | |
| (189) | OH | Cl | N(CH₃)₂ | OH | OH | |
| (190) | OH | Cl | COCH₃ | CH₃ | HCl | |
| (191) | OH | Cl | CH₃ | CH₃ | HCl | |
| (192) | OH | Cl | C₂H₅ | OH₃ | HCl | |
| (193) | OH | Cl | OCH₃ | OH₃ | HCl | |
| (194) | OH | Cl | NO₂ | OH₃ | HCl | |
| (195) | OH | Cl | NH₂ | OH₃ | HCl | |
| (196) | OH | Cl | N(CH₃)₂ | OH₃ | HCl | |
| (197) | OH | F | NH₂ | H | HCl | |
| (198) | OH | F | NH₂ | CH₃ | HCl | |
| (199) | OH | F | NH₂ | H | HCl | |
| (200) | OH | F | NH₂ | OH₃ | HCl | |
| (201) | OH | OH | H | H | HCl | (m.p. |
| | | | | | | 290° C.) |
| (202) | OCH₃ | OCH₃ | H | CO₂CH₃ | pTsOH | |
| (203) | Cl | Cl | CO₂H | H | HCl | |
| (204) | OH₃ | Cl | CH₃ | SCH₃ | HCl | |
| (205) | Cl | Cl | SO₂NH₂ | H | HCl | |

### Examples 206-292

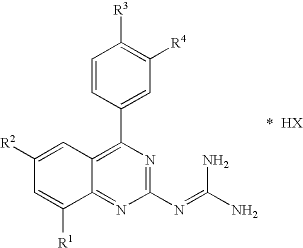

| | R¹ | R² | R³ | R⁴ | HX | |
|---|---|---|---|---|---|---|
| (206) | H | Cl | H | NO₂ | HCl | (m.p. 342° C.) |
| (207) | H | Cl | CH₃ | NO₂ | HCl | |
| (208) | H | Cl | C₂H₅ | NO₂ | HCl | |
| (209) | H | Cl | OCH₃ | NO₂ | HCl | |
| (210) | H | Cl | NO₂ | NO₂ | HCl | |
| (211) | H | Cl | NH₂ | NO₂ | HCl | |
| (212) | H | Cl | N(CH₃)₂ | NO₂ | HCl | |
| (213) | H | Cl | H₂ | NH₂ | HCl | (m.p. 300-340° C.) |
| (214) | H | Cl | CH₃ | NH₂ | HCl | |
| (215) | H | Cl | C₂H₅ | NH₂ | HCl | |
| (216) | H | Cl | OCH₃ | NH₂ | HCl | |
| (217) | H | Cl | NO₂ | NH₂ | HCl | |
| (218) | H | Cl | NH₂ | NH₂ | HCl | |
| (219) | H | Cl | N(CH₃)₂ | NH₂ | HCl | |
| (220) | H | Cl | H | NHCH₃ | HCl | |
| (221) | H | Cl | CH₃ | NHCH₃ | HCl | |
| (222) | H | Cl | C₂H₅ | NHCH₃ | HCl | |
| (223) | H | Cl | OCH₃ | NHCH₃ | HCl | |
| (224) | H | Cl | NO₂ | NHCH₃ | HCl | |
| (225) | H | Cl | NH₂ | NHCH₃ | HCl | |
| (226) | H | Cl | N(CH₃)₂ | NHCH₃ | HCl | |
| (227) | H | Cl | H | N(CH₃)₂ | HCl | |
| (228) | H | Cl | CH₃ | N(CH₃)₂ | HCl | |
| (229) | H | Cl | C₂H₅ | N(OH₃)₂ | HCl | |
| (230) | H | Cl | OCH₃ | N(CH₃)₂ | HCl | |
| (231) | H | Cl | NO₂ | N(OH₃)₂ | HCl | |
| (232) | H | Cl | NH₂ | N(CH₃)₂ | HCl | |
| (233) | H | Cl | N(CH₃) | N(CH₃)₂ | HCl | |
| (234) | H | Cl | H | OH | pTsOH | (m.p. 252-254° C.) |
| (235) | H | Cl | CH₃ | OH | HCl | |
| (236) | H | Cl | C₂H₅ | OH | HCl | |
| (237) | H | Cl | OCH₃ | OH | HCl | |
| (238) | H | Cl | NO₂ | OH | HCl | |
| (239) | H | Cl | NH₂ | OH | HCl | |
| (240) | H | Cl | N(OH₃)₂ | OH | HCl | |
| (241) | H | Cl | CN | CH₃ | HCl | |
| (242) | H | Cl | CH₃ | CH₃ | HCl | |
| (243) | H | Cl | C₂H₅ | CH₃ | HCl | |
| (244) | H | Cl | OCH₃ | CH₃ | HCl | |
| (245) | H | Cl | NO₂ | CH₃ | HCl | |
| (246) | H | Cl | NH₂ | CH₃ | HCl | |
| (247) | H | Cl | N(CH₃)₂ | CH₃ | HCl | |
| (248) | H | Cl | CONH₂ | F | HCl | |
| (249) | H | Cl | NO₂ | F | HCl | |
| (250) | H | H | NH₂ | F | HCl | |
| (251) | H | H | NH₂ | CH₃ | HCl | |
| (252) | H | Cl | SCH₃ | Cl | HCl | |
| (253) | C₆H₅ | H | OH₃ | F | HCl | |
| (254) | CN | Cl | F | F | HCl | |
| (255) | H | Cl | H | CN | HCl | (m.p.350° C.) |
| (256) | H | Br | H | CN | HCl | |
| (257) | H | Br | SOCH₃ | F | HCl | |
| (258) | H | NO₂ | H | F | HCl | |
| (259) | H | OCH₃ | CN | F | HCl | |
| (260) | H | OH | H | F | HCl | |
| (261) | H | NH₂ | H | F | HCl | |
| (262) | H | SCH₃ | H | F | HCl | |
| (263) | H | OH₃ | CONH₂ | F | HCl | |
| (264) | H | C₆H₅ | H | F | HCl | |
| (265) | H | CF₃ | SOCH₃ | F | HCl | |
| (266) | H | OCF₃ | H | F | HCl | |
| (267) | H | CN | H | F | HCl | |
| (268) | H | F | SOCH₃ | F | HCl | |
| (269) | H | SOCH₃ | H | F | HCl | |
| (270) | H | SO₂CH₃ | H | F | HCl | |
| (271) | H | Cl | CN | F | HCl | |
| (272) | H | Cl | CONH₂ | Cl | HCl | |
| (273) | H | Cl | H | OCF₃ | pTSOH | (m.p. 260-264° C.) |
| (274) | H | Cl | OCF₃ | F | HCl | |
| (275) | Cl | Cl | SO₂NH₂ | F | HCl | |
| (276) | Cl | H | NH₂ | F | HCl | |
| (277) | Cl | H | NH₂ | OH₃ | HCl | |
| (278) | CH₃ | Cl | NHCH₃ | F | HCl | |
| (279) | F | Cl | CH₃ | NHCH₃ | HCl | |
| (280) | H | H | C₆H₅ | F | HCl | |
| (281) | Cl | NH₂ | F | F | HCl | |
| (282) | NH₂ | Cl | Cl | F | HCl | |
| (283) | SCH₃ | H | H | F | HCl | |
| (284) | H | F | N(CH₃)₂ | F | HCl | |
| (285) | H | Cl | SCH₃ | F | HCl | |
| (286) | H | H | OCF₃ | CH₃ | HCl | |
| (287) | H | Cl | SOCH₃ | H | HCl | (m.p. 240° C.) |
| (288) | H | Cl | CH₃ | NH₂ | pTsOH | (m.p. 217-218° C.) |
| (289) | H | Cl | H | OCF₃ | HCl | (m.p. 260-264° C.) |
| (290) | H | Cl | H | CO₂CH₃ | HCl | (m.p. 275-277° C.) |
| (291) | H | Cl | CH₃ | NO₂ | pTsOH | (m.p. 218-220° C.) |
| (292) | H | Cl | H | NHCOCH₃ | HCl | (m.p. 317-320° C.) |

### Examples 293-379

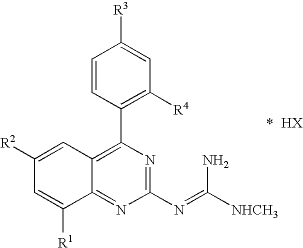

| | R¹ | R² | R³ | R⁴ | HX | |
|---|---|---|---|---|---|---|
| (293) | H | Cl | H | H | pTsOH | (m.p. 268-296° C.) |
| (294) | H | Cl | CH₃ | H | HCl | (m.p. 291-293° C.) |
| (295) | H | Cl | C₂H₅ | H | HCl | |
| (296) | H | Cl | OCH₃ | H | HCl | |
| (297) | H | Cl | NO₂ | H | HCl | |
| (298) | H | Cl | NH₂ | H | HCl | |
| (299) | H | Cl | N(CH₃)₂ | H | HCl | |
| (300) | H | Cl | H | NH₂ | HCl | |
| (301) | H | Cl | CH₃ | NH₂ | HCl | |
| (302) | H | H | H | NH₂ | pTsOH | (m.p. 231-233° C.) |
| (303) | H | Cl | OCH₃ | NH₂ | HCl | |
| (304) | H | Cl | NO₂ | NH₂ | HCl | |
| (305) | H | Cl | NH₂ | NH₂ | HCl | |
| (306) | H | Cl | N(CH₃)₂ | NH₂ | HCl | |
| (307) | H | Cl | H | NHCH₃ | HCl | |
| (308) | H | Cl | CH₃ | NHCH₃ | HCl | |
| (309) | H | Cl | C₂H₅ | NHCH₃ | HCl | |
| (310) | H | Cl | OCH₃ | NHCH₃ | HCl | |
| (311) | H | Cl | NO₂ | NHCH₃ | HCl | |
| (312) | H | Cl | NH₂ | NHCH₃ | HCl | |
| (313) | H | Cl | N(OH₃)₂ | NHCH₃ | HCl | |
| (314) | H | Cl | H | N(CH₃)₂ | HCl | |
| (315) | H | Cl | OH₃ | N(CH₃)₂ | HCl | |
| (316) | H | Cl | C₂H₅ | N(OH₃)₂ | HCl | |
| (317) | H | Cl | OCH₃ | N(OH₃)₂ | HCl | |
| (318) | H | Cl | NO₂ | N(CH₃)₂ | HCl | |
| (319) | H | Cl | NH₂ | N(CH₃)₂ | HCl | |
| (320) | H | Cl | N(OH₃)₂ | N(OH₃)₂ | HCl | |
| (321) | H | Cl | H | OH | HCl | |
| (322) | H | Cl | CH₃ | OH | HCl | |
| (323) | H | Cl | C₂H₅ | OH | HCl | |
| (324) | H | Cl | OCH₃ | OH | HCl | |
| (325) | H | Cl | NO₂ | OH | HCl | |
| (326) | H | Cl | NH₂ | OH | HCl | |
| (327) | H | Cl | N(OH₃)₂ | OH | HCl | |
| (328) | H | Cl | H | CH₃ | HCl | |
| (329) | H | Cl | OH₃ | CH₃ | HCl | |
| (330) | H | Cl | C₂H₅ | CH₃ | HCl | |
| (331) | H | Cl | OCH₃ | CH₃ | HCl | |
| (332) | H | Cl | NO₂ | CH₃ | HCl | |
| (333) | H | Cl | NH₂ | CH₃ | HCl | |
| (334) | H | Cl | N(OH₃)₂ | OH₃ | HCl | |
| (335) | H | Cl | H | NO₂ | pTsOH | (m.p. 278-279° C.) |
| (336) | H | Cl | NO₂ | H | HCl | |
| (337) | H | H | NH₂ | H | HCl | |
| (338) | H | H | NH₂ | CH₃ | HCl | |
| (339) | H | Cl | CH₃ | Cl | HCl | |
| (340) | H | H | CH₃ | H | HCl | |
| (341) | H | Cl | H | F | HCl | |
| (342) | H | Cl | F | H | HCl | |
| (343) | H | Br | H | H | HCl | |
| (344) | H | Br | H | F | HCl | |
| (345) | H | NO₂ | H | H | HCl | |
| (346) | H | OCH₃ | H | H | HCl | |
| (347) | H | OH | H | H | HCl | |
| (348) | H | NH₂ | H | H | HCl | |
| (349) | H | SCH₃ | H | H | HCl | |
| (350) | H | CH₃ | H | H | HCl | |
| (351) | H | C₆H₅ | H | H | HCl | |
| (352) | H | CF₃ | H | H | HCl | |
| (353) | H | OCF₃ | H | H | HCl | |
| (354) | H | CN | H | H | HCl | |
| (355) | H | F | H | H | HCl | |
| (356) | H | SOCH₃ | H | H | HCl | |
| (357) | H | SO₂OH₃ | H | H | HCl | |
| (358) | H | Cl | CN | H | HCl | |
| (359) | H | Cl | H | Cl | HCl | |
| (360) | H | Cl | H | OCF₃ | HCl | |
| (361) | H | Cl | OCF₃ | H | HCl | |
| (362) | Cl | Cl | H | H | HCl | |
| (363) | Cl | H | NH₂ | H | HCl | |
| (364) | Cl | H | NH₂ | CH₃ | HCl | |
| (365) | CH₃ | Cl | CH₃ | H | HCl | |
| (366) | F | Cl | CH₃ | H | HCl | |
| (367) | H | H | H | H | pTsOH | (m.p. 225-226° C.) |
| (368) | Cl | H | H | H | HCl | |
| (369) | H | Cl | Cl | H | HCl | |
| (370) | SCH₃ | H | H | H | HCl | |
| (371) | H | F | CH₃ | H | HCl | |
| (372) | H | Cl | SCH₃ | H | HCl | |
| (373) | CH₃ | H | H | H | HCl | |
| (374) | H | Cl | C₆H₅ | H | HCl | |
| (375) | H | Cl | OH₃ | NO₂ | HCl | |
| (376) | H | H | Br | H | HCl | |
| (377) | H | H | OCH₃ | H | HCl | |
| (378) | H | H | H | NH₂ | HCl | |
| (379) | H | Cl | H | NH₂ | pTsOH | (m.p. 252-254° C.) |

### Examples 380-465

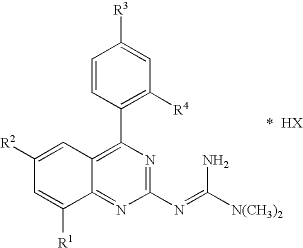

| | R¹ | R² | R³ | R⁴ | HX | |
|---|---|---|---|---|---|---|
| (380) | H | Cl | H | H | pTsOH | (m.p.216-217° C.) |
| (381) | H | Cl | CH₃ | H | pTSOH | (m.p.176-177° C.) |
| (382) | H | Cl | C₂H₅ | H | HCl | |
| (383) | H | Cl | OCH₃ | H | HCl | |
| (384) | H | Cl | NO₂ | H | HCl | |
| (385) | H | Cl | NH₂ | H | HCl | |
| (386) | H | Cl | N(CH₃)₂ | H | HCl | |
| (387) | H | Cl | H | NH₂ | HCl | |
| (388) | H | Cl | OH₃ | NH₂ | HCl | |
| (389) | H | H | H | NH₂ | pTsOH | (m.p.>200° C.) |
| | | | | | | decomposition) |
| (390) | H | Cl | OCH₃ | NH₂ | HCl | |
| (391) | H | Cl | NO₂ | NH₂ | HCl | |
| (392) | H | Cl | NH₂ | NH₂ | HCl | |
| (393) | H | Cl | N(CH₃)₂ | NH₂ | HCl | |
| (394) | H | Cl | H | NHCH₃ | HCl | |
| (395) | H | C1 | CH₃ | NHCH₃ | HCl | |
| (396) | H | Cl | C₂H₅ | NHCH₃ | HCl | |
| (397) | H | Cl | OCH₃ | NHCH₃ | HCl | |
| (398) | H | Cl | NO₂ | NHCH₃ | HCl | |
| (399) | H | Cl | NH₂ | NHCH₃ | HCl | |
| (400) | H | Cl | N(CH₃)₂ | NHCH₃ | HCl | |
| (401) | H | Cl | H | N(CH₃)₂ | HCl | |
| (402) | H | Cl | CH₃ | N(CH₃)₂ | HCl | |
| (403) | H | Cl | C₂H₅ | N(CH₃)₂ | HCl | |
| (404) | H | Cl | OCH₃ | N(CH₃)₂ | HCl | |
| (405) | H | Cl | NO₂ | N(CH₃)₂ | HCl | |
| (406) | H | Cl | NH₂ | N(CH₃)₂ | HCl | |
| (407) | H | Cl | N(CH₃)₂ | N(CH₃)₂ | HCl | |
| (408) | H | Cl | H | OH | HCl | |
| (409) | H | Cl | CH₃ | OH | HCl | |
| (410) | H | Cl | CH₃ | OH | HCl | |
| (411) | H | Cl | OCH₃ | OH | HCl | |
| (412) | H | Cl | NO₂ | OH | HCl | |
| (413) | H | Cl | NH₂ | OH | HCl | |
| (414) | H | Cl | N(CH₃)₂ | OH | HCl | |
| (415) | H | Cl | H | OH₃ | HCl | |
| (416) | H | Cl | OH₃ | CH₃ | HCl | |
| (417) | H | Cl | C₂H₅ | CH₃ | HCl | |
| (418) | H | Cl | OCH₃ | CH₃ | HCl | |
| (419) | H | Cl | NO₂ | CH₃ | HCl | |
| (420) | H | Cl | NH₂ | CH₃ | HCl | |
| (421) | H | Cl | N(CH₃)₂ | CH₃ | HCl | |
| (422) | H | Cl | H | NO₂ | pTsOH | (m.p.233-235° C.) |
| (423) | H | Cl | NO₂ | H | HCl | |
| (424) | H | H | NH₂ | H | HCl | |
| (425) | H | H | NH₂ | CH₃ | HCl | |
| (426) | H | Cl | CH₃ | Cl | HCl | |
| (427) | H | H | CH₃ | H | HCl | |
| (428) | H | Cl | H | F | HCl | |
| (429) | H | Cl | F | H | HCl | |
| (430) | H | Br | H | H | HCl | |
| (431) | H | Br | H | F | HCl | |
| (432) | H | NO₂ | H | H | HCl | |
| (433) | H | OCH₃ | H | H | HCl | |
| (434) | H | OH | H | H | HCl | |
| (435) | H | NH₂ | H | H | HCl | |
| (436) | H | SCH₃ | H | H | HCl | |
| (437) | H | CH₃ | H | H | HCl | |
| (438) | H | C₆H₅ | H | H | HCl | |
| (439) | H | CF₃ | H | H | HCl | |
| (440) | H | OCF₃ | H | H | HCl | |
| (441) | H | CN | H | H | HCl | |
| (442) | H | F | H | H | HCl | |
| (443) | H | SOCH₃ | H | H | HCl | |
| (444) | H | SO₂CH₃ | H | H | HCl | |
| (445) | H | Cl | CN | H | HCl | |
| (446) | H | Cl | H | Cl | HCl | |
| (447) | H | Cl | H | OCF₃ | HCl | |
| (448) | H | Cl | OCF₃ | H | HCl | |
| (449) | Cl | Cl | H | H | HCl | |
| (450) | Cl | H | NH₂ | H | HCl | |
| (451) | Cl | H | NH₂ | CH₃ | HCl | |
| (452) | CH₃ | Cl | CH₃ | H | HCl | |
| (453) | F | Cl | OH₃ | H | HCl | |
| (454) | H | H | H | H | HCl | |
| (455) | Cl | H | H | H | HCl | |
| (456) | H | Cl | Cl | H | HCl | |
| (457) | SCH₃ | H | H | H | HCl | |
| (458) | H | F | OH₃ | H | HCl | |
| (459) | H | Cl | SOH₃ | H | HCl | |
| (460) | CH₃ | H | H | H | HCl | |
| (461) | H | Cl | C₆H₅ | H | HCl | |
| (462) | H | Cl | OH₃ | NO₂ | HCl | |
| (463) | H | H | Br | H | HCl | |
| (464) | H | H | OCH₃ | H | HCl | |
| (465) | H | H | H | NH₂ | HCl | |

### Examples 466-552

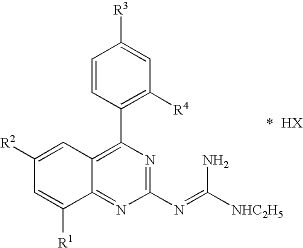

| | R¹ | R² | R³ | R⁴ | HX | |
|---|---|---|---|---|---|---|
| (466) | H | Cl | H | H | pTsOH | (m.p. 236-238° C.) |
| (467) | H | Cl | CH₃ | H | pTsOH | (m.p. 244-246° C.) |
| (468) | H | Cl | C₂H₅ | H | HCl | |
| (469) | H | Cl | OCH₃ | H | HCl | |
| (470) | H | Cl | NO₂ | H | HCl | |
| (471) | H | Cl | NH₂ | H | HCl | |
| (472) | H | Cl | N(CH₃)₂ | H | HCl | |
| (473) | H | Cl | H | NH₂ | HCl | |
| (474) | H | Cl | CH₃ | NH₂ | HCl | |
| (475) | H | H | H | NH₂ | pTsOH | (m.p. >200° C.) |
| | | | | | | decomposition) |
| (476) | H | Cl | OCH₃ | NH₂ | HCl | |
| (477) | H | Cl | NO₂ | NH₂ | HCl | |
| (478) | H | Cl | NH₂ | NH₂ | HCl | |
| (479) | H | Cl | N(CH₃)₂ | NH₂ | HCl | |
| (480) | H | Cl | H | NHCH₃ | HCl | |
| (481) | H | Cl | CH₃ | NHCH₃ | HCl | |
| (482) | H | Cl | C₂H₅ | NHCH₃ | HCl | |
| (483) | H | Cl | OCH₃ | NHCH₃ | HCl | |
| (484) | H | Cl | NO₂ | NHCH₃ | HCl | |
| (485) | H | Cl | NH₂ | NHCH₃ | HCl | |
| (486) | H | Cl | N(CH₃)₂ | NHCH₃ | HCl | |
| (487) | H | Cl | H | N(CH₃)₂ | HCl | |
| (488) | H | Cl | CH₃ | N(CH₃)₂ | HCl | |
| (489) | H | Cl | C₂H₅ | N(CH₃)₂ | HCl | |
| (490) | H | Cl | OCH₃ | N(CH₃)₂ | HCl | |
| (491) | H | Cl | NO₂ | N(CH₃)₂ | HCl | |
| (492) | H | Cl | NH₂ | N(CH₃)₂ | HCl | |
| (493) | H | Cl | N(CH₃)₂ | N(CH₃)₂ | HCl | |
| (494) | H | Cl | H | OH | HCl | |
| (495) | H | Cl | CH₃ | OH | HCl | |
| (496) | H | Cl | C₂H₅ | OH | HCl | |
| (497) | H | Cl | OCH₃ | OH | HCl | |
| (498) | H | Cl | NO₂ | OH | HCl | |
| (499) | H | Cl | NH₂ | OH | HCl | |
| (500) | H | Cl | N(CH₃)₂ | OH | HCl | |
| (501) | H | Cl | H | CH₃ | HCl | |
| (502) | H | Cl | CH₃ | CH₃ | HCl | |
| (503) | H | Cl | C₂H₅ | CH₃ | HCl | |
| (504) | H | Cl | OCH₃ | CH₃ | HCl | |
| (505) | H | Cl | NO₂ | OH₃ | HCl | |
| (506) | H | Cl | NH₂ | OH₃ | HCl | |
| (507) | H | Cl | N(CH₃)₂ | OH₃ | HCl | |
| (508) | H | Cl | H | NO₂ | HCl | |
| (509) | H | Cl | NO₂ | H | HCl | |
| (510) | H | H | NH₂ | H | HCl | |
| (511) | H | H | NH₂ | CH₃ | HCl | |
| (512) | H | Cl | CH₃ | Cl | HCl | |
| (513) | H | H | CH₃ | H | HCl | |
| (514) | H | Cl | H | F | HCl | |
| (515) | H | Cl | F | H | HCl | |
| (516) | H | Br | H | H | HCl | |
| (517) | H | Br | H | F | HCl | |
| (518) | H | NO₂ | H | H | HCl | |
| (519) | H | OCH₃ | H | H | HCl | |
| (520) | H | OH | H | H | HCl | |
| (521) | H | NH₂ | H | H | HCl | |
| (522) | H | SCH_{3 H} | H | HCl | | |
| (523) | H | CH₃ | H | H | HCl | |
| (524) | H | C₆H₅ | H | H | HCl | |
| (525) | H | CF₃ | H | H | HCl | |
| (526) | H | OCF₃ | H | H | HCl | |
| (527) | H | CN | H | H | HCl | |
| (528) | H | F | H | H | HCl | |
| (529) | H | SOCH₃ | H | H | HCl | |
| (530) | H | SO₂CH₃ | H | H | HCl | |
| (531) | H | Cl | CN | H | HCl | |
| (532) | H | Cl | H | Cl | HCl | |
| (533) | H | Cl | H | OCF₃ | HCl | |
| (534) | H | Cl | OCF₃ | H | HCl | |
| (535) | Cl | Cl | H | H | HCl | |
| (536) | Cl | H | NH₂ | H | HCl | |
| (537) | Cl | H | NH₂ | OH₃ | HCl | |
| (538) | CH₃ | Cl | CH₃ | H | HCl | |
| (539) | F | Cl | OH₃ | H | HCl | |
| (540) | H | H | H | H | HCl | |
| (541) | Cl | H | H | H | HCl | |
| (542) | H | Cl | Cl | H | HCl | |
| (543) | SCH₃ | H | H | H | HCl | |
| (544) | H | F | CH₃ | H | HCl | |
| (545) | H | Cl | SCH₃ | H | HCl | |
| (546) | CH₃ | H | H | H | HCl | |
| (547) | H | Cl | C₆H₅ | H | HCl | |
| (548) | H | Cl | CH₃ | NO₂ | HCl | |
| (549) | H | H | Br | H | HCl | |
| (550) | H | H | OCH₃ | H | HCl | |
| (551) | H | H | H | NH₂ | HCl | |
| (552) | H | Cl | H | NH₂ | pTsOH | (m.p. 231-232° C.) |

### Examples 553-639

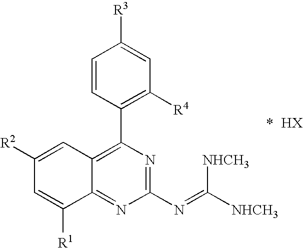

| | R¹ | R² | R³ | R⁴ | HX | |
|---|---|---|---|---|---|---|
| (553) | H | Cl | H | H | pTsOH | |
| (554) | H | Cl | CH₃ | H | HCl | |
| (555) | H | Cl | C₂H₅ | H | HCl | |
| (556) | H | Cl | OCH₃ | H | HCl | |
| (557) | H | Cl | NO₂ | H | HCl | |
| (558) | H | Cl | NH₂ | H | HCl | |
| (559) | H | Cl | N(CH₃)₂ | H | HCl | |
| (560) | H | Cl | H | NH₂ | HCl | (m.p. 298-301° C.) |
| (561) | H | Cl | CH₃ | NH₂ | HCl | |
| (562) | H | Cl | C₂H₅ | NH₂ | HCl | |
| (563) | H | Cl | OCH₃ | NH₂ | HCl | |
| (564) | H | Cl | NO₂ | NH₂ | HCl | |
| (565) | H | Cl | NH₂ | NH₂ | HCl | |
| (566) | H | Cl | N(CH₃)₂ | NH₂ | HCl | |
| (567) | H | Cl | H | NHCH₃ | HCl | |
| (568) | H | Cl | OH₃ | NHCH₃ | HCl | |
| (569) | H | Cl | C₂H₅ | NHCH₃ | HCl | |
| (570) | H | Cl | OCH₃ | NHCH₃ | HCl | |
| (571) | H | Cl | NO₂ | NHCH₃ | HCl | |
| (572) | H | Cl | NH₂ | NHCH₃ | HCl | |
| (573) | H | Cl | N(CH₃)₂ | NHCH₃ | HCl | |
| (574) | H | Cl | H | N(CH₃)₂ | HCl | |
| (575) | H | Cl | CH₃ | N(CH₃)₂ | HCl | |
| (576) | H | Cl | C₂H₅ | N(CH₃)₂ | HCl | |
| (577) | H | Cl | OCH₃ | N(CH₃)₂ | HCl | |
| (578) | H | Cl | NO₂ | N(CH₃)₂ | HCl | |
| (579) | H | Cl | NH₂ | N(CH₃)₂ | HCl | |
| (580) | H | Cl | N(CH₃)₂ | N(CH₃)₂ | HCl | |
| (581) | H | Cl | H | OH | HCl | |
| (582) | H | Cl | OH₃ | OH | HCl | |
| (583) | H | Cl | C₂H₅ | OH | HCl | |
| (584) | H | Cl | OCH₃ | OH | HCl | |
| (585) | H | Cl | NO₂ | OH | HCl | |
| (586) | H | Cl | NH₂ | OH | HCl | |
| (587) | H | Cl | N(CH₃)₂ | OH | HCl | |
| (588) | H | Cl | H | CH₃ | HCl | |
| (589) | H | Cl | CH₃ | CH₃ | HCl | |
| (590) | H | Cl | C₂H₅ | CH₃ | HCl | |
| (591) | H | Cl | OCH₃ | CH₃ | HCl | |
| (592) | H | Cl | NO₂ | CH₃ | HCl | |
| (593) | H | Cl | NH₂ | CH₃ | HCl | |
| (594) | H | Cl | N(CH₃)₂ | OH₃ | HCl | |
| (595) | H | Cl | H | NO₂ | pTsOH | (m.p. 217-220° C.) |
| (596) | H | Cl | NO₂ | H | HCl | |
| (597) | H | H | NH₂ | H | HCl | |
| (598) | H | H | NH₂ | CH₃ | HCl | |
| (599) | H | Cl | CH₃ | Cl | HCl | |
| (600) | H | H | CH₃ | H | HCl | |
| (601) | H | Cl | H | F | HCl | |
| (602) | H | Cl | F | H | HCl | |
| (603) | H | Br | H | H | HCl | |
| (604) | H | Br | H | F | HCl | |
| (605) | H | NO₂ | H | H | HCl | |
| (606) | H | OCH₃ | H | H | HCl | |
| (607) | H | OH | H | H | HCl | |
| (608) | H | NH₂ | H | H | HCl | |
| (609) | H | SCH₃ | H | H | HCl | |
| (610) | H | CH₃ | H | H | HCl | |
| (611) | H | C₆H₅ | H | H | HCl | |
| (612) | H | CF₃ | H | H | HCl | |
| (613) | H | OCF₃ | H | H | HCl | |
| (614) | H | CN | H | H | HCl | |
| (615) | H | F | H | H | HCl | |
| (616) | H | SOCH₃ | H | H | HCl | |
| (617) | H | SO₂CH₃ | H | H | HCl | |
| (618) | H | Cl | CN | H | HCl | |
| (619) | H | Cl | H | Cl | HCl | |
| (620) | H | Cl | H | OCF₃ | HCl | |
| (621) | H | Cl | OCF₃ | H | HCl | |
| (622) | Cl | Cl | H | H | HCl | |
| (623) | Cl | H | NH₂ | H | HCl | |
| (624) | Cl | H | NH₂ | CH₃ | HCl | |
| (625) | CH₃ | Cl | CH₃ | H | HCl | |
| (626) | F | Cl | CH₃ | H | HCl | |
| (627) | H | H | H | H | HCl | |
| (628) | Cl | H | H | H | HCl | |
| (629) | H | Cl | Cl | H | HCl | |
| (630) | SCH₃ | H | H | H | HCl | |
| (631) | H | F | CH₃ | H | HCl | |
| (632) | H | Cl | SCH₃ | H | HCl | |
| (633) | CH₃ | H | H | H | HCl | |
| (634) | H | Cl | C₆H₅ | H | HCl | |
| (635) | H | Cl | CH₃ | NO₂ | HCl | |
| (636) | H | H | Br | H | HCl | |
| (637) | H | H | OCH₃ | H | HCl | |
| (638) | H | Cl | H | NH₂ | pTsOH | |
| (639) | H | Cl | H | NO₂ | HCl | |

### Examples 640-726

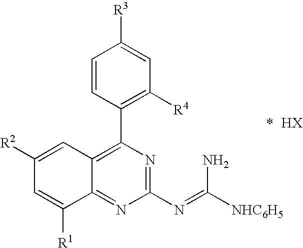

| | R¹ | R² | R³ | R⁴ | HX | |
|---|---|---|---|---|---|---|
| (640) | H | Cl | H | H | HCl | |
| (641) | H | Cl | CH₃ | H | HCl | |
| (642) | H | Cl | C₂H₅ | H | HCl | |
| (643) | H | Cl | OCH₃ | H | HCl | |
| (644) | H | Cl | NO₂ | H | HCl | |
| (645) | H | Cl | NH₂ | H | HCl | |
| (646) | H | Cl | N(CH₃)₂ | H | HCl | |
| (647) | H | Cl | H | NH₂ | pTsOH | (m.p. 178-180° C.) |
| (648) | H | Cl | CH₃ | NH₂ | HCl | |
| (649) | H | Cl | C₂H₅ | NH₂ | HCl | |
| (650) | H | Cl | OCH₃ | NH₂ | HCl | |
| (651) | H | Cl | NO₂ | NH₂ | HCl | |
| (652) | H | Cl | NH₂ | NH₂ | HCl | |
| (653) | H | Cl | N(CH₃)₂ | NH₂ | HCl | |
| (654) | H | Cl | H | NHCH₃ | HCl | |
| (655) | H | Cl | CH₃ | NHCH₃ | HCl | |
| (656) | H | Cl | C₂H₅ | NHCH₃ | HCl | |
| (657) | H | Cl | OCH₃ | NHCH₃ | HCl | |
| (658) | H | Cl | NO₂ | NHCH₃ | HCl | |
| (659) | H | Cl | NH₂ | NHCH₃ | HCl | |
| (660) | H | Cl | N(CH₃)₂ | NHCH₃ | HCl | |
| (661) | H | Cl | H | N(CH₃)₂ | HCl | |
| (662) | H | Cl | CH₃ | N(CH₃)₂ | HCl | |
| (663) | H | Cl | C₂H₅ | N(CH₃)₂ | HCl | |
| (664) | H | Cl | OCH₃ | N(CH₃)₂ | HCl | |
| (665) | H | Cl | NO₂ | N(CH₃)₂ | HCl | |
| (666) | H | Cl | NH₂ | N(CH₃)₂ | HCl | |
| (667) | H | Cl | N(CH₃)₂ | N(CH₃)₂ | HCl | |
| (668) | H | Cl | H | OH | HCl | |
| (669) | H | Cl | CH₃ | OH | HCl | |
| (670) | H | Cl | C₂H₅ | OH | HCl | |
| (671) | H | Cl | OCH₃ | OH | HCl | |
| (672) | H | Cl | NO₂ | OH | HCl | |
| (673) | H | Cl | NH₂ | OH | HCl | |
| (674) | H | Cl | N(CH₃)₂ | OH | HCl | |
| (675) | H | Cl | H | CH₃ | HCl | |
| (676) | H | Cl | CH₃ | CH₃ | HCl | |
| (677) | H | Cl | C₂H₅ | CH₃ | HCl | |
| (678) | H | Cl | OCH₃ | CH₃ | HCl | |
| (679) | H | Cl | NO₂ | CH₃ | HCl | |
| (680) | H | Cl | NH₂ | CH₃ | HCl | |
| (681) | H | Cl | N(CH₃)₂ | CH₃ | HCl | |
| (682) | H | Cl | H | NO₂ | HCl | |
| (683) | H | Cl | NO₂ | H | HCl | |
| (684) | H | H | NH₂ | H | HCl | |
| (685) | H | H | NH₂ | CH₃ | HCl | |
| (686) | H | Cl | CH₃ | Cl | HCl | |
| (687) | H | H | CH₃ | H | HCl | |
| (688) | H | Cl | H | F | HCl | |
| (689) | H | Cl | F | H | HCl | |
| (690) | H | Br | H | H | HCl | |
| (691) | H | Br | H | F | HCl | |
| (692) | H | NO₂ | H | H | HCl | |
| (693) | H | OCH₃ | H | H | HCl | |
| (694) | H | OH | H | H | HCl | |
| (695) | H | NH₂ | H | H | HCl | |
| (696) | H | SCH₃ | H | H | HCl | |
| (697) | H | CH₃ | H | H | HCl | |
| (698) | H | C₆H₅ | H | H | HCl | |
| (699) | H | CF₃ | H | H | HCl | |
| (700) | H | OCF₃ | H | H | HCl | |
| (701) | H | CN | H | H | HCl | |
| (702) | H | F | H | H | HCl | |
| (703) | H | SOCH₃ | H | H | HCl | |
| (704) | H | SO₂CH₃ | H | H | HCl | |
| (705) | H | Cl | CN | H | HCl | |
| (706) | H | Cl | H | Cl | HCl | |
| (707) | H | Cl | H | OCF₃ | HCl | |
| (708) | H | Cl | OCF₃ | H | HCl | |
| (709) | Cl | Cl | H | H | HCl | |
| (710) | Cl | H | NH₂ | H | HCl | |
| (711) | Cl | H | NH₂ | CH₃ | HCl | |
| (712) | CH₃ | Cl | CH₃ | H | HCl | |
| (713) | F | Cl | CH₃ | H | HCl | |
| (714) | H | H | H | H | HCl | |
| (715) | Cl | H | H | H | HCl | |
| (716) | H | Cl | Cl | H | HCl | |
| (717) | SCH₃ | H | H | H | HCl | |
| (718) | H | F | CH₃ | H | HCl | |
| (719) | H | Cl | SCH₃ | H | HCl | |
| (720) | CH₃ | H | H | H | HCl | |
| (721) | H | Cl | C₆H₅ | H | HCl | |
| (722) | H | Cl | CH₃ | NO₂ | HCl | |
| (723) | H | H | Br | H | HCl | |
| (724) | H | H | OCH₃ | H | HCl | |
| (725) | H | Cl | H | NH₂ | pTsOH | (m.p. 178-180° C.) |
| (726) | H | Cl | H | H | pTsOH | (m.p. 219-220° C.) |

### Examples 727-813

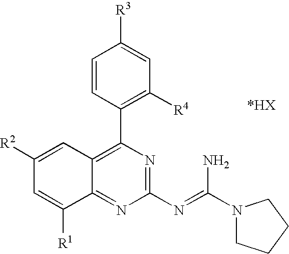

| | R¹ | R² | R³ | R⁴ | HX | |
|---|---|---|---|---|---|---|
| (727) | H | Cl | H | H | HCl | (m.p. |
| | | | | | | 250-252° |
| | | | | | | C.) |
| (728) | H | Cl | CH₃ | H | HCl | |
| (729) | H | Cl | C₂H₅ | H | HCl | |
| (730) | H | Cl | OCH₃ | H | HCl | |
| (731) | H | Cl | NO₂ | H | HCl | |
| (732) | H | Cl | NH₂ | H | HCl | |
| (733) | H | Cl | N(CH₃)₂ | H | HCl | |
| (734) | H | Cl | H | NH₂ | pTsOH | |
| (735) | H | Cl | CH₃ | NH₂ | HCl | |
| (736) | H | Cl | C₂H₅ | NH₂ | HCl | |
| (737) | H | Cl | OCH₃ | NH₂ | HCl | |
| (738) | H | Cl | NO₂ | NH₂ | HCl | |
| (739) | H | Cl | NH₂ | NH₂ | HCl | |
| (740) | H | Cl | N(CH₃)₂ | NH₂ | HCl | |
| (741) | H | Cl | H | NHCH₃ | HCl | |
| (742) | H | Cl | CH₃ | NHCH₃ | HCl | |
| (743) | H | Cl | C₂H₅ | NHCH₃ | HCl | |
| (744) | H | Cl | OCH₃ | NHCH₃ | HCl | |
| (745) | H | Cl | NO₂ | NHCH₃ | HCl | |
| (746) | H | Cl | NH₂ | NHCH₃ | HCl | |
| (747) | H | Cl | N(CH₃)₂ | NHCH₃ | HCl | |
| (748) | H | Cl | H | N(CH₃)₂ | HCl | |
| (749) | H | Cl | CH₃ | N(CH₃)₂ | HCl | |
| (750) | H | Cl | C₂H₅ | N(CH₃)₂ | HCl | |
| (751) | H | Cl | OCH₃ | N(CH₃)₂ | HCl | |
| (752) | H | Cl | NO₂ | N(CH₃)₂ | HCl | |
| (753) | H | Cl | NH₂ | N(CH₃)₂ | HCl | |
| (754) | H | Cl | N(CH₃)₂ | N(CH₃)₂ | HCl | |
| (755) | H | Cl | H | OH | HCl | |
| (756) | H | Cl | CH₃ | OH | HCl | |
| (757) | H | Cl | C₂H₅ | OH | HCl | |
| (758) | H | Cl | OCH₃ | OH | HCl | |
| (759) | H | Cl | NO₂ | OH | HCl | |
| (760) | H | Cl | NH₂ | OH | HCl | |
| (761) | H | Cl | N(CH₃)₂ | OH | HCl | |
| (762) | H | Cl | H | CH₃ | HCl | |
| (763) | H | Cl | CH₃ | CH₃ | HCl | |
| (764) | H | Cl | C₂H₅ | CH₃ | HCl | |
| (765) | H | Cl | OCH₃ | CH₃ | HCl | |
| (766) | H | Cl | NO₂ | CH₃ | HCl | |
| (767) | H | Cl | NH₂ | CH₃ | HCl | |
| (768) | H | Cl | N(CH₃)₂ | CH₃ | HCl | |
| (769) | H | Cl | H | NO₂ | pTsOH | (m.p. |
| | | | | | | 221-224° |
| | | | | | | C.) |
| (770) | H | Cl | NO₂ | H | HCl | |
| (771) | H | H | NH₂ | H | HCl | |
| (772) | H | H | NH₂ | CH₃ | HCl | |
| (773) | H | Cl | CH₃ | Cl | HCl | |
| (774) | H | H | CH₃ | H | HCl | |
| (775) | H | Cl | H | F | HCl | |
| (776) | H | Cl | F | H | HCl | |
| (777) | H | Br | H | H | HCl | |
| (778) | H | Br | H | F | HCl | |
| (779) | H | NO₂ | H | H | HCl | |
| (780) | H | OCH₃ | H | H | HCl | |
| (781) | H | OH | H | H | HCl | |
| (782) | H | NH₂ | H | H | HCl | |
| (783) | H | SCH₃ | H | H | HCl | |
| (784) | H | CH₃ | H | H | HCl | |
| (785) | H | C₆H₅ | H | H | HCl | |
| (786) | H | CF₃ | H | H | HCl | |
| (787) | H | OCF₃ | H | H | HCl | |
| (788) | H | CN | H | H | HCl | |
| (789) | H | F | H | H | HCl | |
| (790) | H | SOCH₃ | H | H | HCl | |
| (791) | H | SO₂CH₃ | H | H | HCl | |
| (792) | H | Cl | CN | H | HCl | |
| (793) | H | Cl | H | Cl | HCl | |
| (794) | H | Cl | H | OCF₃ | HCl | |
| (795) | H | Cl | OCF₃ | H | HCl | |
| (796) | Cl | Cl | H | H | HCl | |
| (797) | Cl | H | NH₂ | H | HCl | |
| (798) | Cl | H | NH₂ | CH₃ | HCl | |
| (799) | CH₃ | Cl | CH₃ | H | HCl | |
| (800) | F | Cl | CH₃ | H | HCl | |
| (801) | H | H | H | H | HCl | |
| (802) | Cl | H | H | H | HCl | |
| (803) | H | Cl | Cl | H | HCl | |
| (804) | SCH₃ | H | H | H | HCl | |
| (805) | H | F | CH₃ | H | HCl | |
| (806) | H | Cl | SCH₃ | H | HCl | |
| (807) | CH₃ | H | H | H | HCl | |
| (808) | H | Cl | C₆H₅ | H | HCl | |
| (809) | H | Cl | CH₃ | NO₂ | HCl | |
| (810) | H | H | Br | H | HCl | |
| (811) | H | H | OCH₃ | H | HCl | |
| (812) | H | Cl | H | NO₂ | HCl | |
| (813) | H | Cl | H | H | pTsOH | |

### Examples 814-900

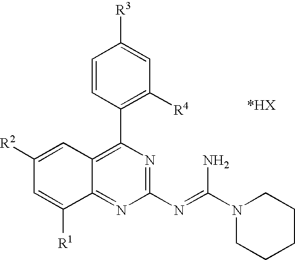

| | R¹ | R² | R³ | R⁴ | HX | |
|---|---|---|---|---|---|---|
| (814) | H | Cl | H | H | HCl | |
| (815) | H | Cl | CH₃ | H | HCl | |
| (816) | H | Cl | C₂H₅ | H | HCl | |
| (817) | H | Cl | OCH₃ | H | HCl | |
| (818) | H | Cl | NO₂ | H | HCl | |
| (819) | H | Cl | NH₂ | H | HCl | |
| (820) | H | Cl | N(CH₃)₂ | H | HCl | |
| (821) | H | Cl | H | NH₂ | pTsOH | |
| (822) | H | Cl | CH₃ | NH₂ | HCl | |
| (823) | H | Cl | C₂H₅ | NH₂ | HCl | |
| (824) | H | Cl | OCH₃ | NH₂ | HCl | |
| (825) | H | Cl | NO₂ | NH₂ | HCl | |
| (826) | H | Cl | NH₂ | NH₂ | HCl | |
| (827) | H | Cl | N(CH₃)₂ | NH₂ | HCl | |
| (828) | H | Cl | H | NHCH₃ | HCl | |
| (829) | H | Cl | CH₃ | NHCH₃ | HCl | |
| (830) | H | Cl | C₂H₅ | NHCH₃ | HCl | |
| (831) | H | Cl | OCH₃ | NHCH₃ | HCl | |
| (832) | H | Cl | NO₂ | NHCH₃ | HCl | |
| (833) | H | Cl | NH₂ | NHCH₃ | HCl | |
| (834) | H | Cl | N(CH₃)₂ | NHCH₃ | HCl | |
| (835) | H | Cl | H | N(CH₃)₂ | HCl | |
| (836) | H | Cl | CH₃ | N(CH₃)₂ | HCl | |
| (837) | H | Cl | C₂H₅ | N(CH₃)₂ | HCl | |
| (838) | H | Cl | OCH₃ | N(CH₃)₂ | HCl | |
| (839) | H | Cl | NO₂ | N(CH₃)₂ | HCl | |
| (840) | H | Cl | NH₂ | N(CH₃)₂ | HCl | |
| (841) | H | Cl | N(CH₃)₂ | N(CH₃)₂ | HCl | |
| (842) | H | Cl | H | OH | HCl | |
| (843) | H | Cl | CH₃ | OH | HCl | |
| (844) | H | Cl | C₂H₅ | OH | HCl | |
| (845) | H | Cl | OCH₃ | OH | HCl | |
| (846) | H | Cl | NO₂ | OH | HCl | |
| (847) | H | Cl | NH₂ | OH | HCl | |
| (848) | H | Cl | N(CH₃)₂ | OH | HCl | |
| (849) | H | Cl | H | CH₃ | HCl | |
| (850) | H | Cl | CH₃ | CH₃ | HCl | |
| (851) | H | Cl | C₂H₅ | CH₃ | HCl | |
| (852) | H | Cl | OCH₃ | CH₃ | HCl | |
| (853) | H | Cl | NO₂ | CH₃ | HCl | |
| (854) | H | Cl | NH₂ | CH₃ | HCl | |
| (855) | H | Cl | N(CH₃)₂ | CH₃ | HCl | |
| (856) | H | Cl | H | NO₂ | HCl | (m.p. |
| | | | | | | 118-120° |
| | | | | | | C.) |
| (857) | H | Cl | NO₂ | H | HCl | |
| (858) | H | H | NH₂ | H | HCl | |
| (859) | H | H | NH₂ | CH₃ | HCl | |
| (860) | H | Cl | CH₃ | Cl | HCl | |
| (861) | H | H | CH₃ | H | HCl | |
| (862) | H | Cl | H | F | HCl | |
| (863) | H | Cl | F | H | HCl | |
| (864) | H | Br | H | H | HCl | |
| (865) | H | Br | H | F | HCl | |
| (866) | H | NO₂ | H | H | HCl | |
| (867) | H | OCH₃ | H | H | HCl | |
| (868) | H | CH | H | H | HCl | |
| (869) | H | NH₂ | H | H | HCl | |
| (870) | H | SCH₃ | H | H | HCl | |
| (871) | H | CH₃ | H | H | HCl | |
| (872) | H | C₆H₅ | H | H | HCl | |
| (873) | H | CF₃ | H | H | HCl | |
| (874) | H | OCF₃ | H | H | HCl | |
| (875) | H | CN | H | H | HCl | |
| (876) | H | F | H | H | HCl | |
| (877) | H | SOCH₃ | H | H | HCl | |
| (878) | H | SO₂CH₃ | H | H | HCl | |
| (879) | H | Cl | CN | H | HCl | |
| (880) | H | Cl | H | Cl | HCl | |
| (881) | H | Cl | H | OCF₃ | HCl | |
| (882) | H | Cl | OCF₃ | H | HCl | |
| (883) | Cl | Cl | H | H | HCl | |
| (884) | Cl | H | NH₂ | H | HCl | |
| (885) | Cl | H | NH₂ | CH₃ | HCl | |
| (886) | CH₃ | Cl | CH₃ | H | HCl | |
| (887) | F | Cl | CH₃ | H | HCl | |
| (888) | H | H | H | H | HCl | |
| (889) | Cl | H | H | H | HCl | |
| (890) | H | Cl | Cl | H | HCl | |
| (891) | SCH₃ | H | H | H | HCl | |
| (892) | H | F | CH₃ | H | HCl | |
| (893) | H | Cl | SCH₃ | H | HCl | |
| (894) | CH₃ | H | H | H | HCl | |
| (895) | H | Cl | C₆H₅ | H | HCl | |
| (896) | H | Cl | CH₃ | NO₂ | HCl | |
| (897) | H | H | Br | H | HCl | |
| (898) | H | H | OCH₃ | H | HCl | |
| (899) | H | Cl | H | NO₂ | HCl | (m.p. |
| | | | | | | 118-120° |
| | | | | | | C.) |
| (900) | H | Cl | H | H | pTsOH | (m.p. |
| | | | | | | >242° C.,) |
| | | | | | | decom- |
| | | | | | | position) |

### Examples 901-961

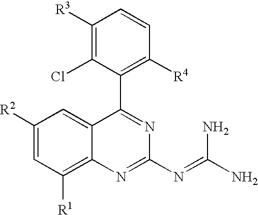

| | R¹ | R² | R³ | R⁴ | HX | |
|---|---|---|---|---|---|---|
| (901) | H | Cl | Cl | NH₂ | pTsOH | (m.p. |
| | | | | | | 322-325° C.) |
| (902) | H | Cl | Cl | NO₂ | pTsOH | (m.p. |
| | | | | | | 220-222° C.) |
| (903) | H | Cl | H | SO₂CH₃ | pTsOH | |
| (904) | H | Cl | CH₃ | SO₂CH₃ | HCl | |
| (905) | H | Cl | C₂H₅ | SO₂CH₃ | HCl | |
| (906) | H | Cl | OCH₃ | SO₂CH₃ | HCl | |
| (907) | H | Cl | NO₂ | H | HCl | |
| (908) | H | Cl | NH₂ | H | pTsOH | |
| (909) | H | Cl | N(CH₃)₂ | H | pTsOH | |
| (910) | H | Cl | H | NH₂ | HCl | |
| (911) | H | Cl | CH₃ | NH₂ | pTsOH | |
| (912) | H | Cl | C₂H₅ | NH₂ | HCl | |
| (913) | H | Cl | OCH₃ | NH₂ | HCl | |
| (914) | H | Cl | NO₂ | NH₂ | HCl | |
| (915) | H | Cl | NH₂ | NH₂ | HCl | |
| (916) | H | Cl | N(CH₃)₂ | NH₂ | HCl | |
| (917) | H | Cl | H | NHCH₃ | HCl | |
| (918) | H | Cl | CH₃ | NHCH₃ | HCl | |
| (919) | H | Cl | C₂H₅ | NHCH₃ | HCl | |
| (920) | H | Cl | OCH₃ | NHCH₃ | HCl | |
| (921) | H | Cl | NO₂ | NHCH₃ | HCl | |
| (922) | H | Cl | NH₂ | NHCH₃ | HCl | |
| (923) | H | Cl | N(CH₃)₂ | NHCH₃ | HCl | |
| (924) | H | Cl | N(CH₃)₂ | NHCH₃ | HCl | |
| (925) | H | Cl | H | N(CH₃)₂ | HCl | |
| (926) | H | Cl | CH₃ | N(CH₃)₂ | HCl | |
| (927) | H | Cl | C₂H₅ | N(CH₃)₂ | HCl | |
| (928) | H | Cl | OCH₃ | N(CH₃)₂ | HCl | |
| (929) | H | Cl | NO₂ | N(CH₃)₂ | HCl | |
| (930) | H | Cl | NH₂ | N(CH₃)₂ | HCl | |
| (931) | H | Cl | N(CH₃)₂ | N(CH₃)₂ | HCl | |
| (932) | H | Cl | H | OH | HCl | |
| (933) | H | Cl | OH₃ | OH | HCl | |
| (934) | H | Cl | C₂H₅ | OH | HCl | |
| (935) | H | Cl | OCH₃ | OH | HCl | |
| (936) | H | Cl | NO₂ | OH | HCl | |
| (937) | H | Cl | NH₂ | OH | HCl | |
| (938) | H | Cl | N(CH₃)₂ | OH | HCl | |
| (939) | H | Cl | SO₂CH₃ | CH₃ | HCl | |
| (940) | H | Cl | H | CN | HCl | |
| (941) | H | Cl | C₂H₅ | SO₂NH₂ | HCl | |
| (942) | H | Cl | OCF₃ | CH₃ | HCl | |
| (943) | H | Cl | NO₂ | CH₃ | HCl | |
| (944) | H | Cl | NH₂ | CH₃ | HCl | |
| (945) | H | Cl | N(CH₃)₂ | CH₃ | HCl | |
| (946) | H | Cl | H | NO₂ | pTsOH | |
| (947) | H | Cl | NO₂ | H | HCl | |
| (948) | H | H | NH₂ | H | HCl | |
| (949) | H | H | NH₂ | CH₃ | HCl | |
| (950) | H | Cl | CH₃ | CO—NH₂ | HCl | |
| (951) | H | H | CH₃ | SO₂CH₃ | pTsOH | |
| (952) | H | Cl | OH | F | pTsOH | |
| (953) | H | Cl | F | SCH₃ | HCl | |
| (954) | H | Br | H | CONH₂ | pTsOH | |
| (955) | H | Br | CO—NH₂ | F | pTsOH | |
| (956) | H | NO₂ | H | H | pTsOH | |
| (957) | H | OCH₃ | H | OCF₃ | pTsOH | |
| (958) | H | OH | H | H | HCl | |
| (959) | H | NH₂ | H | H | HCl | |
| (960) | H | SCH₃ | H | H | HCl | |
| (961) | H | CH₃ | CN | CO—NH₂ | pTsOH | |

Pharmacological Tests

The method used for the characterisation of the compounds of the formula I as NHE-3 inhibitors is described below.

The compounds of the formula I were characterised with respect to their selectivity for the NHE-1 to NHE-3 isoforms. The three isoforms were expressed in stable form in mouse fibroblast cell lines. The inhibitory action of the compounds was assessed by determination of the EIPA-sensitive take-up of ²²Na⁺ into the cells after intracellular acidosis.

Material and Methods

LAP1 Cell Lines Which Express the Different NHE Isoforms

The LAP1 cell lines which express the NHE-1, -2 and -3 isoforms (a mouse fibroblast cell line) was obtained from Prof. J. Pouysségur (Nice, France). The transfection was carried out by the method of Franchi et al. (1986). The cells were cultivated in Dulbeccos modified eagle medium (DMEM) with 10% of deactivated foetal calf serum (FCS). For selection of the NHE-expressing cells, the so-called “acid killing method” of Sardet et al. (1989) was used. The cells were firstly incubated for 30 minutes in an NH₄Cl-containing bicarbonate- and sodium-free buffer. The extracellular NH₄Cl was then removed by washing with a bicarbonate-, NH₄Cl- and sodium-free buffer. The cells were subsequently incubated in a bicarbonate-free NaCl-containing buffer. Only those cells which functionally express NHE were able to survive in the intracellular acidification to which they were subjected.

Characterisation of NHE Inhibitors with Respect to Their Isoform Selectivity

With the above-mentioned mouse fibroblast cell lines which express the NHE-1, NHE-2 and NHE-3 isoforms, compounds were tested for selectivity with respect to the isoforms by the procedure described by Counillon et al. (1993) and Scholz et al. (1995). The cells were acidified intracellularly by the NH₄Cl prepulse method and subsequently by incubation in a bicarbonate-free ²²Na⁺-containing buffer. Owing to the intracellular acidification, NHE was activated, and sodium was taken up into the cells. The effect of the test compound was expressed as inhibition of EIPA (ethylisopropylamiloride)-sensitive ²²Na⁺ take-up.

The cells which expressed NHE-1, NHE-2 and NHE-3 were sown out in a density of 5-7.5×10⁴ cells/Nell in 24-well microtitre plates and cultured to confluence for from 24 to 48 hours. The medium was removed by suction, and the cells were incubated for 60 minutes at 37° C. in NH₄Cl buffer (50 mM NH₄Cl, 70 mM choline chloride, 15 mM MOPS, pH 7.0). The buffer was subsequently removed, and the cells were rapidly covered twice with the choline chloride wash buffer (120 mM choline chloride, 15 MM PIPES/tris, 0.1 mM ouabain, 1 mM MgCl₂, 2 mM CaCl₂, pH 7.4) and filtered off with suction. The cells were subsequently covered with the choline chloride charging buffer (120 mM choline chloride, 15 mM PIPES/tris, 0.1 mM PIPES/tris, 0.1 mM ouabain, 1 mM MgCl₂, 2 mM CaCl₂, pH 7.4, ²²Na^{±} (0.925 kBg/100 ml of charging buffer)) and then incubated in this buffer for 6 minutes. After expiry of the incubation time, the incubation buffer was removed by suction. In order to remove extracellular radioactivity, the cells were washed rapidly four times with ice-cold phosphate-buffered saline solution (PBS). The cells were then solubilised by addition of 0.3 ml of 0.1 N NaOH per well. The cell fragment-containing solutions were transferred into scintillation tubes. Each well was then washed twice with 0.3 ml of 0.1 N NaOH, and the washing solutions were likewise introduced into the corresponding scintillation tubes. Scintillation cocktail was added to the tubes containing the cell lysate, and the radioactivity taken up into the cells was determined by determination of the β radiation.

### LITERATURE

Counillon et al. (1993) Mol. Pharmacol. 44: 1041-1045

J. Membrane Biol. 120, 41-49

Franchi et al. (1986) Proc. Natl. Aced. Sci. USA 83: 9388-9392

J. Membrane Bid. 118, 193-214

Sardet et al. (1989) Cell 56: 271-280

Scholz et al. (1995) Cardiovasc. Res. 29: 260-268

The examples below relate to pharmaceutical preparations:

### Example A: Injection Vials

A solution of 100 g of an NHE-3 inhibitor of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions Each injection vial contains 5 mg of active ingredient.

### Example B: Suppositories

A mixture of 20 g of an NHE-3 inhibitor of the formula I is melted with 100 g of soya lecithin and 1400 g of cocoa butter, poured into moulds and allowed to cool. Each suppository contains 20 mg of active ingredient.

### Example C: Solution

A solution is prepared from 1 g of an NHE-3 inhibitor of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D: Ointment

500 mg of an NHE-3 inhibitor of the formula I are mixed with 99.5 g of Vaseline under aseptic conditions.

### Example E: Tablets

A mixture of 1 kg of an NHE-3 inhibitor of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F: Coated Tablets

Tablets are pressed analogously to Example E and subsequently coated in a conventional manner with a coating of sucrose, potato starch, talc, tragacanth and dye.

### Example G: Capsules

2 kg of an NHE-3 inhibitor of the formula I are introduced into hard gelatine capsules in a conventional manner in such a way that each capsule contains 20 mg of the active ingredient.

### Example H: Ampoules

A solution of 1 kg of an NHE-3 inhibitor of the formula I in 60 l of bidistilled water is sterile filtered, transferred into ampoules, lyophilised under sterile conditions and sealed under sterile conditions. Each ampoule contains 10 mg of active ingredient.

1. Compounds of the formulae 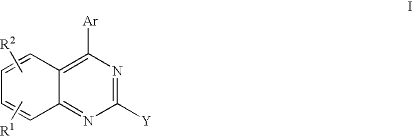
in which
Y is 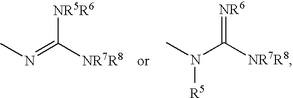
Ar is phenyl or naphthyl, each of which is unsubstituted or monosubstituted by R³ and/or R⁴,
R¹, R², R³ and R⁴ are each, independently of one another, H, A, OA, Hal, CF₃, OH, NO₂, NH₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO-NA₂, SO₂NH₂, SO₂NHA₇ SO₂NA₂, or phenyl which is unsubstituted or monosubstituted or poly-substituted by A, OA, Hal or CF₃,
A is alkyl having 1, 2, 3, 4, 5 or 6 carbon atoms
Hal is F. Cl, Br or I
R⁵, R⁶, R⁷ and R⁸ are each, independently of one another, H, A, or phenyl which is unsubstituted or monosubstituted or polysubstituted by A, OA, Hal or CF₃, where R⁵ and R⁷, R⁵ and R⁶, and R⁷ and R⁸ are able to form 5-7-membered rings,
and their salts and solvates, with the proviso that compounds in which R⁵, R⁶, R⁷ and R⁸ are simultaneously H and none of the radicals R¹, R², R³ and R⁴ is OH, NO₂, NH₁₂, NHA, NA₂, NH—CO-A, NH—CO-Ph, SA, SO-A, SO₂-A, SO₂-Ph, CN, OCF₃, CO-A, CO₂H, CO₂A, CO—NH₂, CO—NHA, CO—NA₂, SO₂NH₂, SO₂NHA, SO₂NA₂, or phenyl which is unsubstituted or monosubstituted or polysubstituted by A, OA, Hal or CF₃, are excluded.

2. Compounds of the formula I according to claim 1 and their salts and solvates as NHE-3 inhibitors.

3. Compounds of the formula I according to claim 1 and their physiologically acceptable salts or solvates for use in combating illnesses.

4. Use of compounds of the formula I according to claim 1 and/or their physiologically acceptable salts or solvates for the preparation of a medicament.

5. Use of compounds of the formula I according to claim 1 and their physiologically acceptable salts and/or solvates for the preparation of a medicament for the treatment of hypertonia, thrombosis, ischaemic states of the heart, of the peripheral and central nervous system and of strokes, ischaemic states of peripheral organs and limbs, and for the treatment of shock states.

6. Use of compounds of the formula I according to claim 1 and their physiologically acceptable salts and/or solvates for the preparation of a medicament for use in surgical operations and organ transplants and for the preservation and storage of transplants for surgical measures.

7. Use of compounds of the formula I according to claim 1 and their physiologically acceptable salts and/or solvates for the preparation of a medicament for the treatment of illnesses in which cell proliferation represents a primary or secondary cause, for the treatment or prophylaxis of disorders of fat metabolism or disturbed breathing drive.

8. Use of compounds of the formula I according to claim 1 and their physiologically acceptable salts and/or solvates for the preparation of a medicament for the treatment of renal ischaemia, ischaemic intestinal illnesses or for the prophylaxis of acute or chronic renal illnesses.

9. Use of compounds of the formula I according to claim 1 and their physiologically acceptable salts and/or solvates for the preparation of a medicament for the treatment of bacterial and parasitic illnesses.

10. Pharmaceutical preparation characterised by a content of at least one NHE-3 inhibitor according to claim 1 and/or one of its physiologically acceptable salts and/or solvates.

11. Compound selected from the group consisting of the compounds I1 to I10:

| | |
|---|---|
| N-(6-chloro-4-phenylquinazolin-2-yl)-N′-methylguanidine | I1 |
| N-(6-chloro-4-p-tolylquinazolin-2-yl)-N′-methylguanidine | I2 |
| N-[6-chloro-4-(2-nitrophenyl)quinazolin-2-yl]-N′-methyl- | I3 |
| guanidine | |
| N-[4-(2-aminophenyl)-6-chloroquinazolin-2-yl]-N′-methyl- | I4 |
| guanidine | |
| N-[6-chloro-4-(4-methyl-2-nitrophenyl)quinazolin-2-yl]-N′- | I5 |
| methylguanidine | |
| N-[4-(2-amino-4-methylphenyl)-6-chloroquinazolin-2-yl]-N′- | I6 |
| methylguanidine | |
| N-[6-chloro-4-(2-nitrophenyl)quinazolin-2-yl]guanidine | I7 |
| N-[4-(2-aminophenyl)-6-chloroquinazolin-2-yl]guanidine | I8 |
| N-[6-chloro-4-(4-methyl-2-nitrophenyl)quinazolin-2-yl]- | I9 |
| guanidine | |
| N-[4-(2-amino-4-methylphenyl)-6-chloroquinazolin-2-yl]- | I10 |
| guanidine | |

and their salts and solvates.

12. Compounds according to claim 1 as medicament active ingredients.

13. Process for the preparation of the 2-guanidino-4-arylquinazolines of the formula I and their salts and solvates, characterised in that either
(a)
compounds of the formula II 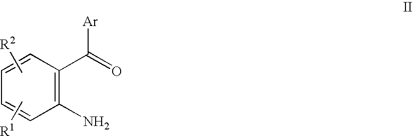
in which R¹, R² and Ar are as defined above, are reacted with 1-cyanoguanidine or a correspondingly N-alkylated or N-arylated cyanoguanidine of the formula NC—Y, in which Y is as defined in claim 1, or
(b)
instead of a compound of the formula NC—Y, a compound of the formula III
HN═CX—Y III
in which X is —S-alkyl, —S-aryl, —O-alkyl or —O-aryl, is reacted with a compound of the formula II, or
(c)
2-chloro-4-arylquinazolines of the formula IV 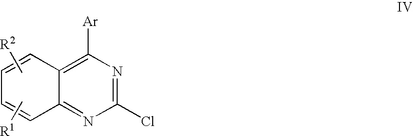
in which Ar R¹ and R² are as defined in claim 1,
are reacted with a compound of the formula HY, in which Y is as defined in claim 1,
and optionally, after steps (a), (b) or (c), a basic or acidic compound of the formula I is converted into one of its salts or solvates by treatment with an acid or base.
